(19) United States
(12) Patent Application Publication
(10) Pub. No.: US 2015/0108409 A1
(43) Pub. Date: Apr. 23, 2015

(54) SMALL MOLECULES AND THEIR USE AS ORGANIC SEMICONDUCTORS

(71) Applicant: Merck Patent GmbH, Darmstadt (DE)

(72) Inventors: Frank Egon Meyer, Winchester (GB); Nicolas Blouin, Southampton (GB); Toby Cull, Romsey (GB); William Mitchell, Chandler's Ford (GB)

(73) Assignee: Merck Patent GmbH, Darmstadt (DE)

(21) Appl. No.: 14/405,431

(22) PCT Filed: May 6, 2013
(86) PCT No.: PCT/EP2013/001334

§ 371 Date: Dec. 4, 2014

(30) Foreign Application Priority Data
Jun. 5, 2012 (EP) 12004283.3

(51) Int. Cl. H01L 51/00

(52) U.S. Cl. CPC H01L 51/0068; H01L 51/0074; H01L 51/5012
USPC 252/500; 548/126

(57) ABSTRACT

The invention relates to compounds based on benzo[1,2-b:4,5-b′]dithiophene (BDT), methods for their preparation and intermediates used therein, mixtures and formulations containing them, the use of the compounds, mixtures and formulations as semiconductor in organic electronic (OE) devices, especially in organic photovoltaic (OPV) devices, and to OE devices comprising these compounds, mixtures or formulations.

### FIELD OF THE INVENTION

The invention relates to compounds based on benzo[1,2-b:4,5-b′]dithiophene (BDT), methods for their preparation and intermediates used therein, mixtures and formulations containing them, the use of the compounds, mixtures and formulations as semiconductor in organic electronic (OE) devices, especially in organic photovoltaic (OPV) devices, and to OE devices comprising these compounds, mixtures or formulations.

### BACKGROUND AND PRIOR ART

In recent years there has been growing interest in the use of organic semiconductors, including conjugated polymers and small molecules, for various electronic applications.

One particular area of importance is the field of organic photovoltaics (OPV). Organic semiconductors (OSCs) have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Numerous small molecules have been developed for solution processable OPV devices as disclosed for example in Thuc-Quyen Nguyen et al., Chem. Mater. 2011, 23, 470-482. However, device power conversion efficiency is still generally low. Two recent examples have demonstrated an important step towards higher power conversion efficiencies: squarine based small molecules combined with C₇₀ fullerenes have shown a power conversion efficiency of 5.2% in a solution processed OPV device as disclosed in Stephen R. Forrest et al., Adv. Ener. Mater. 2011, 1, 184-187, and DPP based small molecules combined with PCBM-C60 fullerenes have shown a power conversion efficiency of 4.1% in a solution processed OPV device as disclosed in Loser S. et al.; J. Am. Chem. Soc. 2011, 133, 8142-8145.

Another particular area of importance is the field of organic field effect transistors (OFETs) including as a sub-class organic thin film transistors (OTFTs), which are used for example in RFID tags or in backplanes of liquid crystal displays. Compared to the classical, Si-based FETs, OFETs can be fabricated much more cost-effectively by solution coating methods such as spin-coating, drop casting, dip-coating, and more efficiently, ink-jet printing. Solution processing of OSCs requires the molecular materials to be soluble enough in non-toxic solvents, stable in the solution state, easy to crystallise when solvents are evaporated, and provide high charge carrier mobility with low off current.

However, the OSC materials that have been suggested in prior art for use in OPV devices do still suffer from certain drawbacks. For example many polymers suffer from limited solubility in commonly used organic solvents, which can inhibit their suitability for device manufacturing methods based on solution processing, or show only limited power conversion efficiency in OPV bulk-hetero-junction devices, or have only limited charge carrier mobility, or are difficult to synthesize and require synthesis methods which are unsuitable for mass production.

In case of OSC materials for OFETs, the currently available OSC materials do also still have some major drawbacks, like a low photo and environment stability particularly in solution states, and a low temperature of the phase transition and melting point. Also for future OLED backplane applications, which demand higher source and drain current, the mobility and processibility of currently available materials needs further improvement.

Therefore, there is still a need for organic semiconducting (OSC) materials that are easy to synthesize, especially by methods suitable for mass production, show good structural organization and film-forming properties, exhibit good electronic properties, especially a high charge carrier mobility, good processibility, especially a high solubility in organic solvents, and high stability in air.

For use in OPV cells, there is a need for OSC materials having a low bandgap, which enable improved light harvesting by the photoactive layer and can lead to higher cell efficiencies, compared to materials of prior art. For use in OFETs, there is a need for OSC materials that show good electronic properties, especially high charge carrier mobility, good processability and high thermal and environmental stability, especially a high solubility in organic solvents.

It was an aim of the present invention to provide compounds for use as organic semiconducting materials that do not have the drawbacks of prior art materials as described above, are easy to synthesize, especially by methods suitable for mass production, and do especially show advantageous properties, especially for OPV and OFET use, as described above. Another aim of the invention was to extend the pool of OSC materials available to the expert. Other aims of the present invention are immediately evident to the expert from the following detailed description.

The inventors of the present invention have found that one or more of the above aims can be achieved by providing monomeric compounds (small molecules) containing a benzo[1,2-b:4,5-b′]dithiophene (BDT) core that is substituted with one or more linear or branched aliphatic hydrocarbyl groups.

It was found that these compounds show good processability and high solubility in organic solvents, and are thus especially suitable for large scale production using solution processing methods. At the same time, they show a low bandgap, high charge carrier mobility, high external quantum efficiency in BHJ solar cells, good morphology when used in p/n-type blends e.g. with fullerenes, high oxidative stability, and are promising materials for organic electronic OE devices, especially for OPV devices with high power conversion efficiency.

Organic semiconductive small molecules comprising BDT moieties have been disclosed in prior art. Chen, Y. et al.; Adv. Mater. 2011, 23, 5387-5391 discloses small molecules with an unsubstituted BDT moiety for use in solar cells. G. C. Bazan et al; J. Am. Chem. Soc., 2012, 134, 3766-3779 discloses alkoxyl substituted BDT moiety for use in solar cells without application data. US 2011/049477 A1 (DuPont) discloses benzene substituted BDT small molecules for use as electroactive material. WO 2011/161262 A1 (Heliatek) discloses small molecules with two terminal dicyanovinyl groups, which may inter alia also comprise BDT moieties, and further discloses their use as evaporable organic semiconductive material for example in photovoltaic applications. However, the above-mentioned documents do neither disclose nor suggest the compounds as claimed hereinafter.

### SUMMARY

The invention relates to compounds of formula I

R^{t1}-(Ar¹)ₐ-(Ar²)_{b}-[(Ar³)_{c}-(Ar⁴)_{d}-U-(Ar⁵)ₑ-(Ar⁶)_{f}]ₙ-(Ar⁷)_{g}-(Ar⁸)ₕ-R^{t2} I

wherein
U is a divalent group of the following structure

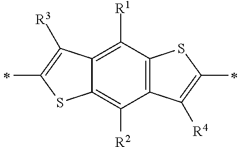

Ar¹⁻⁸ independently of each other denote —CY¹═CY²—, —C≡C—, or aryl or heteroaryl that has 5 to 30 ring atoms and is unsubstituted or substituted by one or more groups R or R¹, and one or more of Ar¹⁻⁸ may also denote U, and wherein those of Ar¹⁻⁸ that are directly adjacent to a group U are different from phenyl and naphthyl,
Y¹, Y² independently of each other denote H, F, Cl or CN,
R¹⁻⁴ independently of each other denote H, F, Cl, —CN, CF₃, R, —CF₂—R, —S—R, —SO₂—R, —SO³—R—C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)—NRR′, —NR′—C(O)—R, —CR′═CR″R′″,
R is alkyl with 1 to 30 C atoms which is straight-chain, branched or cyclic, and is unsubstituted, substituted with one or more F or Cl atoms or CN groups, or perfluorinated, and in which one or more C atoms are optionally replaced by —O—, —S—, —C(O)—, —C(S)—, —SiR⁰R⁰⁰—, —NR⁰R⁰⁰—, —CHR⁰═CR⁰⁰— or —C≡C— such that O- and/or S-atoms are not directly linked to each other,
R⁰, R⁰⁰ independently of each other denote H or C₁₋₁₀ alkyl,
R′, R″, R′″ independently of each other have one of the meanings of R or denote H,
R^{t1,t2} independently of each other denote H, F, Cl, Br, —CN, —CF₃, R, —CF₂—R, —O—R, —S—R, —SO₂—R, —SO³—R—C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)NRR′, —NR′—C(O)—R, —NHR, —NR′R′, —CR′═CR″R′″, —C≡C—R′, —C≡C—SiR′R″R′″, —SiR′R″R′″, —CH═C(CN)—C(O)—OR, —CH═C(COOR)₂, CH═C(CONRR′)₂, CH═C(CN)(Ar⁹),

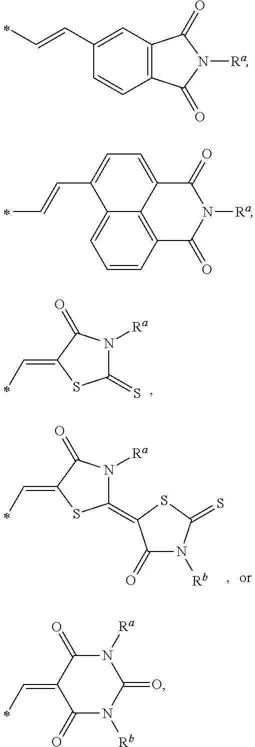

R^{a}, R^{b} are independently of each other aryl or heteroaryl, each having from 4 to 30 ring atoms and being unsubstituted or substituted with one or more groups R or R¹,
Ar⁹ is aryl or heteroaryl, each having from 4 to 30 ring atoms and being unsubstituted or substituted with one or more groups R or R¹
a-h are independently of each other 0 or 1, with at least one of a-h being 1,
n is 1, 2 or 3.

The invention further relates to methods of preparing compounds of formula I and to educts and intermediates used therein.

The invention further relates to the use of compounds of formula I as organic semiconductor in organic electronic (OE) devices, preferably as electron donor in a semiconducting or photoactive material for use in OE devices.

The invention further relates to mixtures comprising one or more compounds of formula I as electron donor component, and further comprising one or more compounds having electron acceptor properties.

The invention further relates to mixtures comprising one or more compounds of formula I, and one or more compounds having one or more properties selected from semiconducting, photoactive, charge transport, hole transport, electron transport, hole blocking, electron blocking, electrically conducting, photoconducting or light emitting properties.

The invention further relates to formulations comprising one or more compounds of formula I or mixtures as described above, and further comprising one or more solvents, preferably selected from organic solvents. The invention further relates to formulations comprising one or more compounds of formula I or mixtures as described above, optionally comprising one or more solvents, preferably selected from organic solvents, and further comprising one or more organic binders or precursors thereof, preferably having a permittivity ∈ at 1,000 Hz and 20° C. of 3.3 or less.

The invention further relates to the use of compounds of formula I, mixtures and formulations as described above and below as charge transport, semiconducting, photoactive, electrically conducting, photoconducting or light emitting material in optical, electrooptical, electronic, electroluminescent or photoluminescent devices, or in components of such devices, or in assemblies comprising such devices or components.

The invention further relates to charge transport, semiconducting, photoactive, electrically conducting, photoconducting or light emitting materials comprising a compound of formula I, a mixture, or a formulation as described above and below.

The invention further relates to optical, electrooptical, electronic, electroluminescent or photoluminescent devices, or components thereof, or assemblies comprising them, which comprise a compound of formula I, a mixture, or a formulation as described above and below, or comprise a charge transport, semiconducting, electrically conducting, photoconducting or light emitting material as described above and below.

The optical, electrooptical, electronic, electroluminescent and photoluminescent devices include, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, laser diodes, Schottky diodes, photoconductors and photodetectors.

Preferred devices include bulk heterojunction (BHJ) OPV devices and inverse BHJ OPV devices.

The components of the above devices include, without limitation, charge injection layers, charge transport layers, interlayers, planarising layers, antistatic films, polymer electrolyte membranes (PEM), conducting substrates and conducting patterns.

The assemblies comprising such devices or components include, without limitation, integrated circuits (IC), radio frequency identification (RFID) tags or security markings or security devices containg them, flat panel displays or backlights thereof, electrophotographic devices, electrophotographic recording devices, organic memory devices, sensor devices, biosensors and biochips.

In addition the compounds, mixtures and formulations of the present invention can be used as electrode materials in batteries and in components or devices for detecting and discriminating DNA sequences.

### DETAILED DESCRIPTION OF THE INVENTION

The compounds of formula I are especially suitable as (electron) donor in p-type semiconducting materials or mixtures, and for the preparation of mixtures of p-type and n-type semiconductors which are useful for application in BHJ or inverse BHJ OPV devices.

The compound of formula I is preferably blended with a further n-type semiconductor like for example a fullerene, for example selected from PCBM-C₆₁, PCBM-C₇₁, bis-PCBM-C₆₁, bis-PCBM-C₇₁ and ICBA, a graphene, or a metal oxide, for example selected from ZnOₓ, TiOₓ, ZTO, MoOₓ and NiOₓ, to form the photoactive layer in the OPV device. The OPV device typically further comprises a first transparent or semi-transparent electrode on a transparent or semi-transparent substrate on one side of the photoactive layer, and a second metallic or semi-transparent electrode on the other side of the photoactive layer. Additional buffer layers can be inserted between the photoactive layer and a specific electrode, wherein these additional buffer layers are acting as hole blocking layer, hole transporting layer, electron blocking layer and/or electron transporting layer, and are comprising for example a metal oxide like for example ZnOₓ, TiOₓ, ZTO, MoO_{X} or NiO_{X}, LiF, a salt like for example LiF or NaF, a conjugated polymer electrolyte like for example PEDOT:PSS, a conjugated polymer like for example PTAA, or an organic compound like for example NPB, Alq₃ or TPD. The compounds of formula I demonstrate the following properties:
i) They have a well defined structure and end-groups (R^{t1,t2}) leading to lower elemental impurity profile (such as palladium, phosphine, tin, halogen and boron) compared to BDT polymer materials as disclosed in prior art, thus enhancing the material lifetime.
ii) They have a well defined structure leading to lower defect incorporation than in the material from the polymerisation reaction as described in prior art, therefore enhancing the material lifetime and molecular organisation.
iii) Additional solubility can be introduced into the organic semiconductor by the inclusion of groups Ar¹⁻⁸ that contain solubilising groups.
iv) Additional solubility can further be introduced into the organic semiconductor by the inclusion of groups R¹⁻⁴ and R^{t1,t2} containing solubilising groups.
v) The benzo[1,2-b:4,5-b′]dithiophene core has a planar structure that enables strong pi-pi stacking in the solid state leading to better improved charge transport properties in the form of higher charge carrier mobility.
vi) Additional fine-tuning of the electronic energies (HOMO/LUMO levels) can be achieved by careful selection of the Ar¹⁻⁸ groups on each side of the benzo[1,2-b:4,5-b′]dithiophene core.

The compounds of formula I are easy to synthesize and exhibit several advantageous properties, like a low bandgap, a high charge carrier mobility, a high solubility in organic solvents, a good processability for the device manufacture process, a high oxidative stability and a long lifetime in electronic devices.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. (see also U.S.

Environmental Protection Agency, 2009, Glossary of technical terms, http://www.epa.gov/oust/cat/TUMGLOSS.HTM, or “Glossary of terms used in physical organic chemistry (IUPAC recommendations 1994)” in Pure and Applied Chemistry, 1994, 66, 1077, pages 1109-1110).

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a small molecule or a polymer) that contains mainly C atoms with sp²-hybridisation (or optionally also sp-hybridisation), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, which may lead to interruption of the conjugation, is still regarded as a conjugated compound.

As used herein, the term “carbyl group” will be understood to mean any monovalent or multivalent organic radical moiety which comprises at least one carbon atom either without any non-carbon atoms (like for example —C≡C—), or optionally combined with at least one non-carbon atom such as N, O, S, P, Si, Se, As, Te or Ge (for example carbonyl etc.). The term “hydrocarbyl group” will be understood to mean a carbyl group that does additionally contain one or more H atoms and optionally contains one or more hetero atoms like for example N, O, S, P, Si, Se, As, Te or Ge.

As used herein, the term “hetero atom” will be understood to mean an atom in an organic compound that is not a H- or C-atom, and preferably will be understood to mean N, O, S, P, Si, Se, As, Te or Ge.

A carbyl or hydrocarbyl group comprising a chain of 3 or more C atoms may be straight-chain, branched and/or cyclic, including spiro and/or fused rings.

Preferred carbyl and hydrocarbyl groups include alkyl, alkoxy, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from N, O, S, P, Si, Se, As, Te and Ge.

The carbyl or hydrocarbyl group may be a saturated or unsaturated acyclic group, or a saturated or unsaturated cyclic group. Unsaturated acyclic or cyclic groups are preferred, especially aryl, alkenyl and alkynyl groups (especially ethynyl). Where the C₁-C₄₀ carbyl or hydrocarbyl group is acyclic, the group may be straight-chain or branched. The C₁-C₄₀ carbyl or hydrocarbyl group includes for example: a C₁-C₄₀ alkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₂-C₂₀ alkenyl group, a C₂-C₂₀ alkynyl group, a C₃-C₂₀ allyl group, a C₄-C₂₀ alkyldienyl group, a C₆-C₁₂ aryl group, and a C₄-C₂₀ polyenyl group, respectively. Also included are combinations of groups having carbon atoms and groups having hetero atoms, like e.g. an alkynyl group, preferably ethynyl, that is substituted with a silyl group, preferably a trialkylsilyl group.

The terms “aryl” and “heteroaryl” as used herein preferably mean a mono-, bi- or tricyclic aromatic or heteroaromatic group with 4 to 30 ring C atoms that may also comprise condensed rings and is optionally substituted with one or more groups L, wherein L is selected from halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, P-Sp-, optionally substituted silyl, or carbyl or hydrocarbyl with 1 to 40 C atoms that is optionally substituted and optionally comprises one or more hetero atoms, and is preferably alkyl, alkoxy, thiaalkyl, alkylcarbonyl, alkoxycarbonyl or alkoxycarbonyloxy with 1 to 20 C atoms that is optionally fluorinated, and R⁰, R⁰⁰, X⁰, P and Sp have the meanings given above and below.

Very preferred substituents L are selected from halogen, most preferably F, or alkyl, alkoxy, oxaalkyl, thioalkyl, fluoroalkyl and fluoroalkoxy with 1 to 12 C atoms or alkenyl, alkynyl with 2 to 12 C atoms.

Especially preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene preferably 2-thiophene, selenophene, preferably 2-selenophene, thieno[3,2-b]thiophene, indole, isoindole, benzofuran, benzothiophene, benzodithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, all of which can be unsubstituted, mono- or polysubstituted with L as defined above. Further examples of heteroaryl groups are those selected from the following formulae An alkyl or alkoxy radical, i.e. where the terminal CH₂ group is replaced by —O—, can be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, nonoxy, decoxy, undecoxy, dodecoxy, tridecoxy or tetradecoxy, for example.

An alkenyl group, wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e. where one CH₂ group is replaced by —O—, is preferably straight-chain 2-oxapropyl (=methoxymethyl), 2- (=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example. Oxaalkyl, i.e. where one CH₂ group is replaced by —O—, is preferably straight-chain 2-oxapropyl (=methoxymethyl), 2- (=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxy-ethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxy-carbonylmethyl, propoxycarbonylmethyl, butoxycarbonylmethyl, 2-(methoxycarbonyl)ethyl, 2-(ethoxycarbonyl)ethyl, 2-(propoxy-carbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e. where one CH₂ group is replaced by —S—, is preferably straight-chain thiomethyl (—SCH₃), 1-thioethyl (—SCH₂CH₃), 1-thiopropyl (=—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is preferably perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, in particular 1,1-difluoroalkyl, all of which are straight-chain or branched.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-propylpentyl, in particular 2-methylbutyl, 2-methylbutoxy, 2-methylpentoxy, 3-methylpentoxy, 2-ethyl-hexoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-meth-oxyoctoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloropropionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment of the present invention, R¹⁻⁴ are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

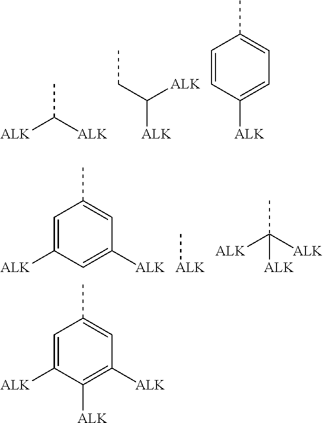

wherein “ALK” denotes optionally fluorinated, preferably linear, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

—CY¹¹═CY¹²— is preferably —CH═CH—, —CF═CF— or —CH═C(CN)—.

As used herein, “halogen” includes F, Cl, Br or I, preferably F, Cl or Br.

A used herein, —CO—, —C(═O)— and —C(O)— will be understood to mean a carbonyl group, i.e. a group having the structure

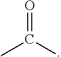

Another aspect of the invention relates to educts and intermediates for the preparation of compounds of formula I, which are selected of formula II

R⁵-(Ar¹⁰)ᵢ—U-(Ar¹¹)ₖ-R⁶ II

wherein U is as defined in formula I,
Ar¹⁰, Ar¹¹ independently of each other, and on each occurrence identically or differently, have one of the meanings of Ar¹ as given in formula I, or one of the preferred meanings as described above and below,
i, k are independently of each other 0, 1, 2 or 3, with i+k>0, and
R⁵, R⁶ are independently of each other a leaving group, preferably selected from the group consisting of H, F, Br, Cl, I, —CH₂Cl, —CHO, —CR^{a}═CR^{b}₂, —SiR^{a}R^{b}R^{c}, —SiR^{a}X′X″, —SiR^{a}R^{b}X′, —SnR^{a}R^{b}R^{c}, —BR^{a}R^{b}, B(OH)₂, —B(OZ²)₂, —O—SO₂Z¹, O-tosylate, O-triflate, O-mesylate, O-nonaflate, —SiMe₂F, —SiMeF₂, —CZ³═C(Z³)₂, —C≡CH, —C≡CSi(Z¹)₃, —ZnX′ and —Sn(Z⁴)₃, wherein X′ and X″ denote halogen, preferably Cl, Br or I, R^{a}, R^{b} and R^{c} independently of each other denote H or alkyl with 1 to 20 C atoms, two of R^{a}, R^{b} and R^{c} may also form an aliphatic ring together with the hetero atom to which they are attached, Z¹⁻⁴ are selected from the group consisting of alkyl and aryl, each being optionally substituted, and two groups Z² may also together form a cyclic group.

In the compounds of formula I and II, Ar¹⁻⁸ and Ar¹⁰⁻¹¹ are selected such that they form a fully conjugated core group together with the group U. In the compounds of formula I the groups R¹⁻⁴ can be selected to improve the properties of the compound, e.g. by increasing the solubility. In the compounds of formula II reactive sites are introduced by the groups R⁵ and R⁶ for use in aryl-aryl coupling reactions.

Preferably Ar¹⁻¹¹ in formula I and II independently of each other, and on each occurrence identically or differently, denote aryl or heteroaryl, which preferably has 5 to 30 ring atoms and is unsubstituted or substituted, preferably by one or more groups R¹ as defined above, or denote U.

Further preferred are compounds of formula I wherein one or more of Ar¹⁻¹¹ are selected from aryl or heteroaryl groups having electron donor properties.

Further preferred are compounds of formula I wherein one or more of Ar¹⁻¹¹ are selected from aryl or heteroaryl groups having electron acceptor properties.

Further preferred are compounds of formula I comprising one or more groups Ar¹⁻¹¹ selected from aryl or heteroaryl groups having electron donor properties, and further comprising one or more groups Ar¹⁻¹¹ selected from aryl or heteroaryl groups having electron acceptor properties.

Very preferred are compounds of formula I, wherein Ar¹⁻⁸ are selected from aryl or heteroaryl having electron donor properties, selected from the group consisting of the following formulae

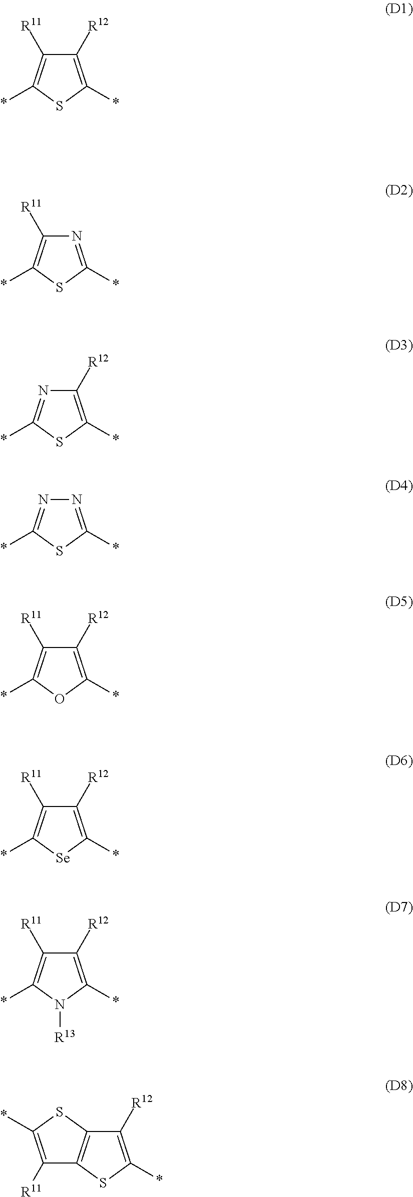

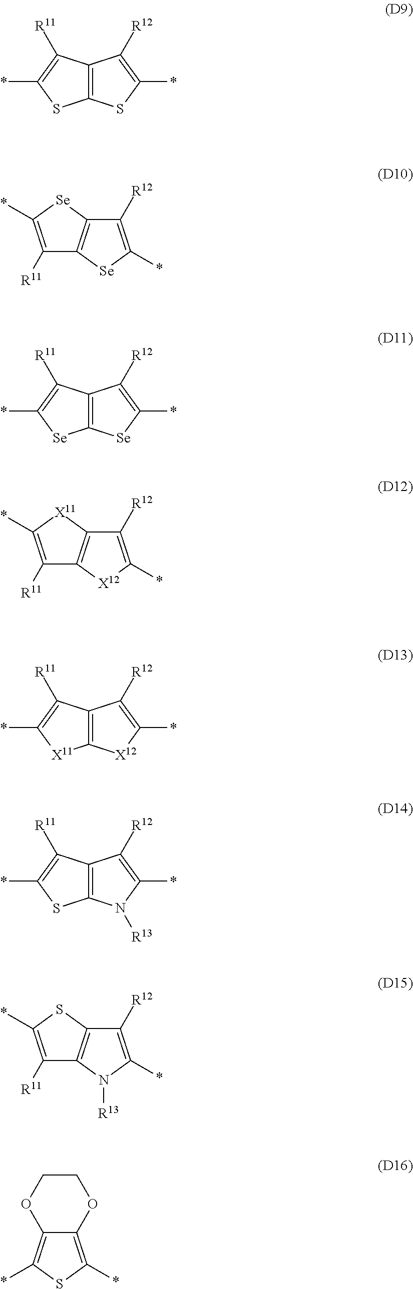

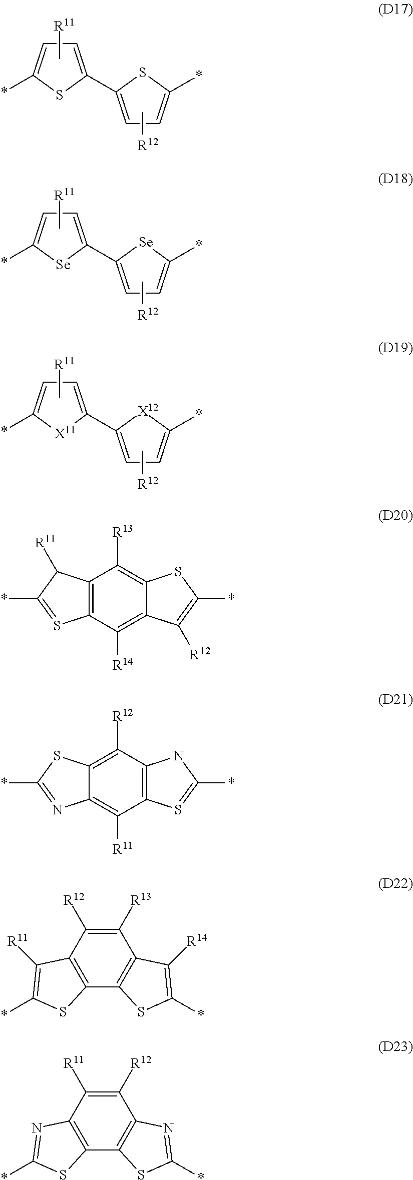

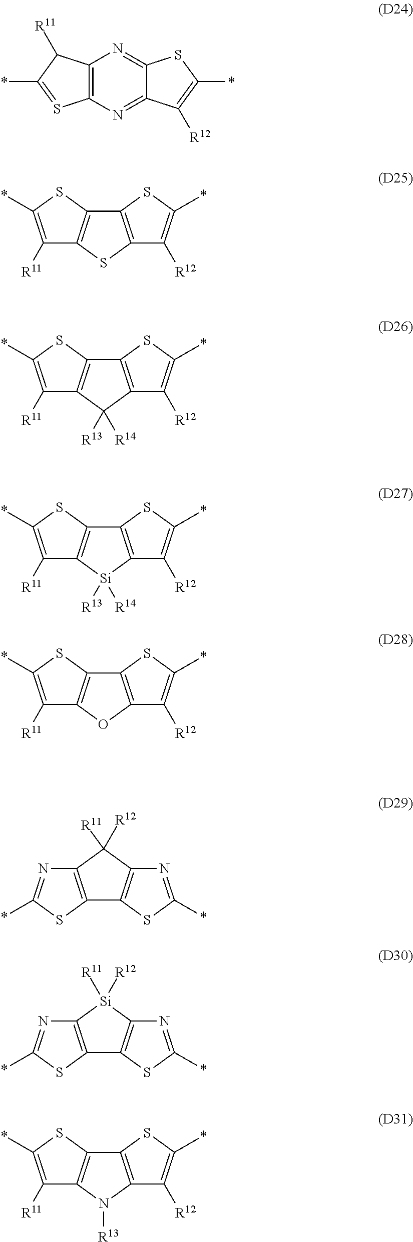

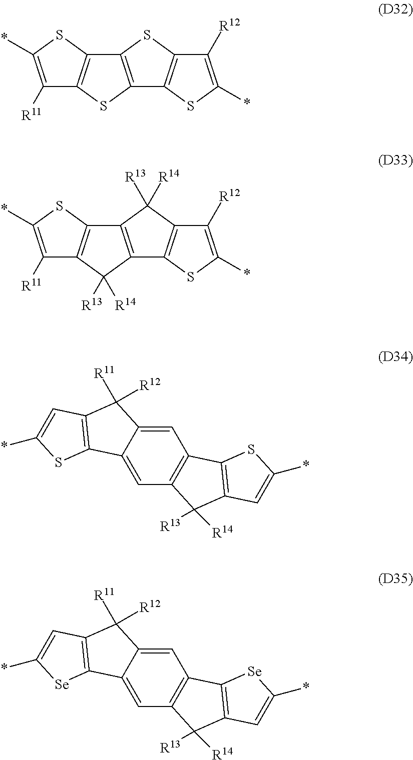

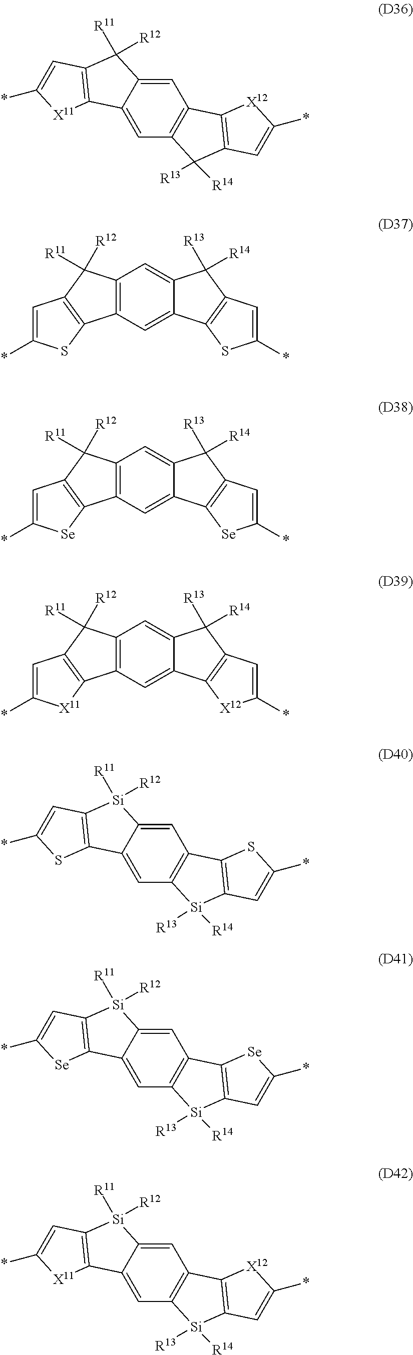

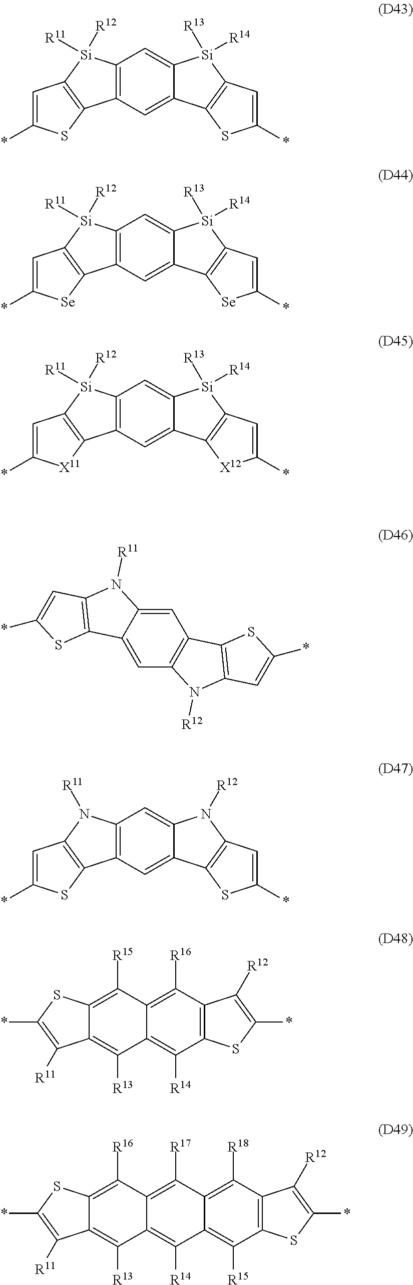

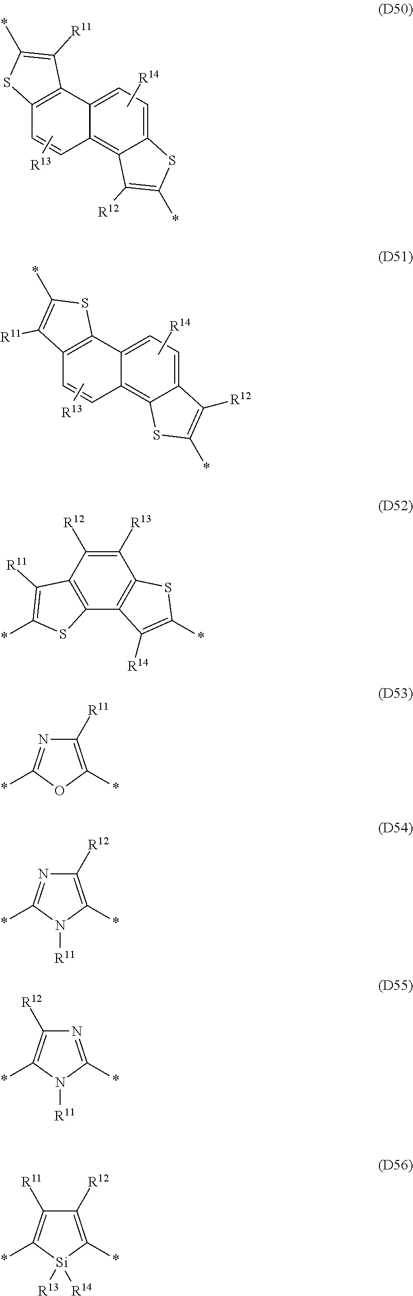

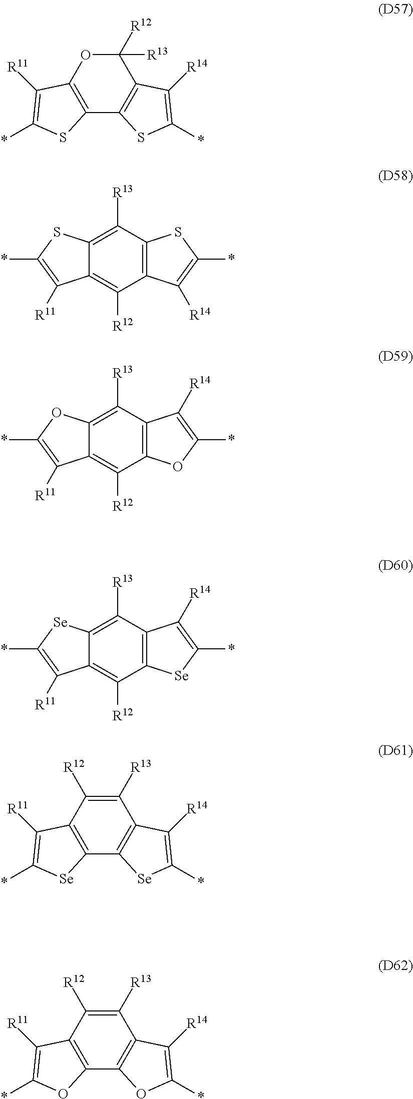

wherein one of X¹¹ and X¹² is S and the other is Se, and R¹¹, R¹², R¹³, R¹⁴, R¹⁵, R¹⁶, R¹⁷ and R¹⁸ independently of each other denote H or have one of the meanings of R¹ as defined above and below.

Further preferred are compounds of formula I, wherein Ar¹⁻⁸ are selected from aryl or heteroaryl having electron acceptor properties, selected from the group consisting of the following formulae

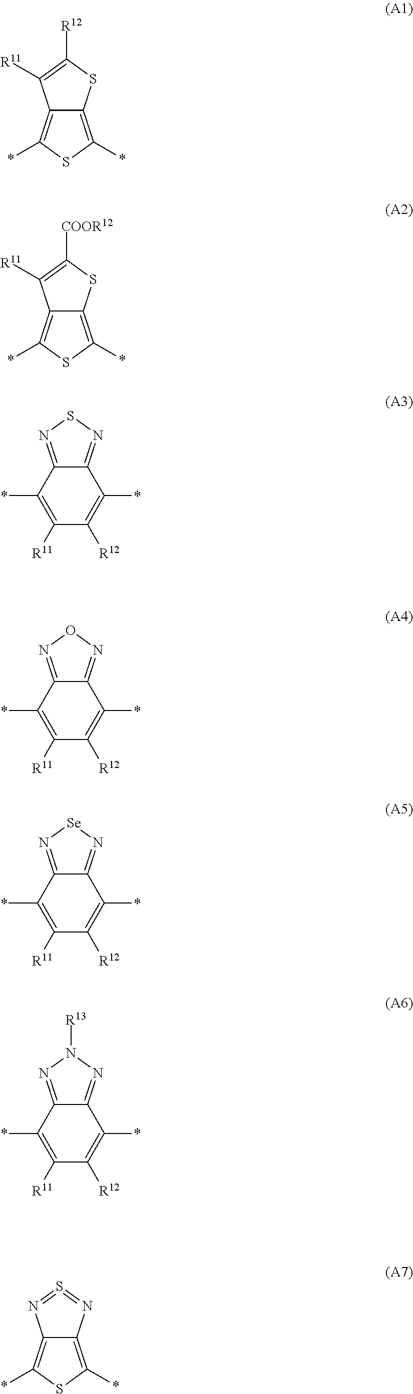

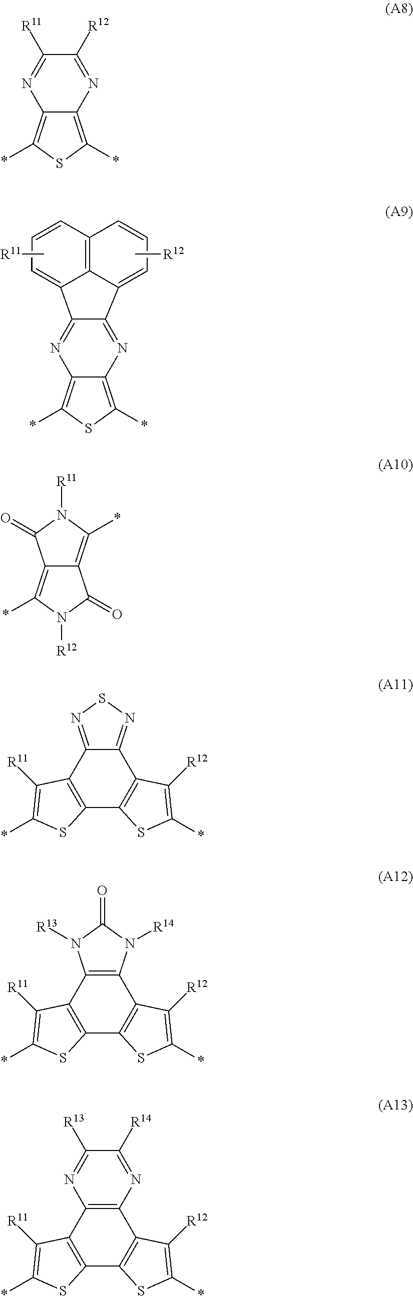

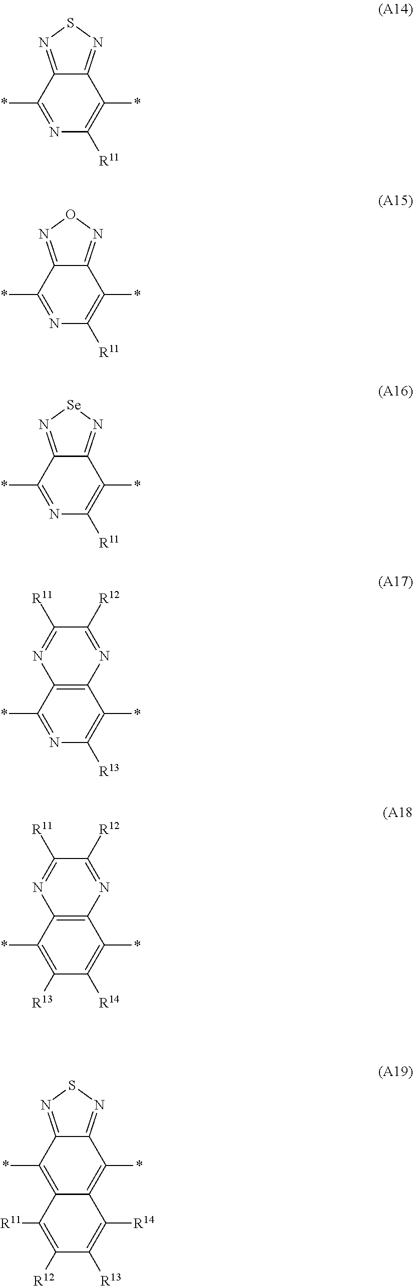

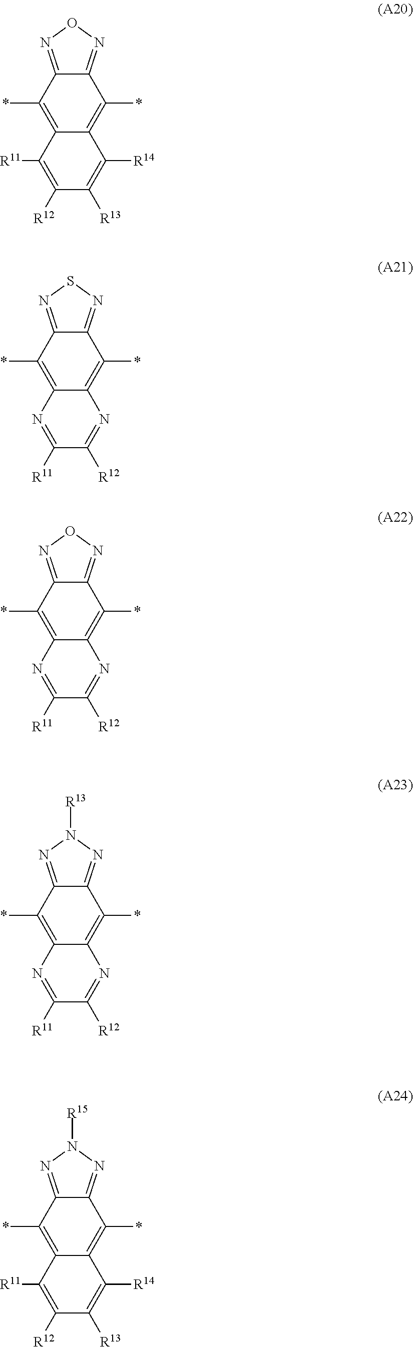

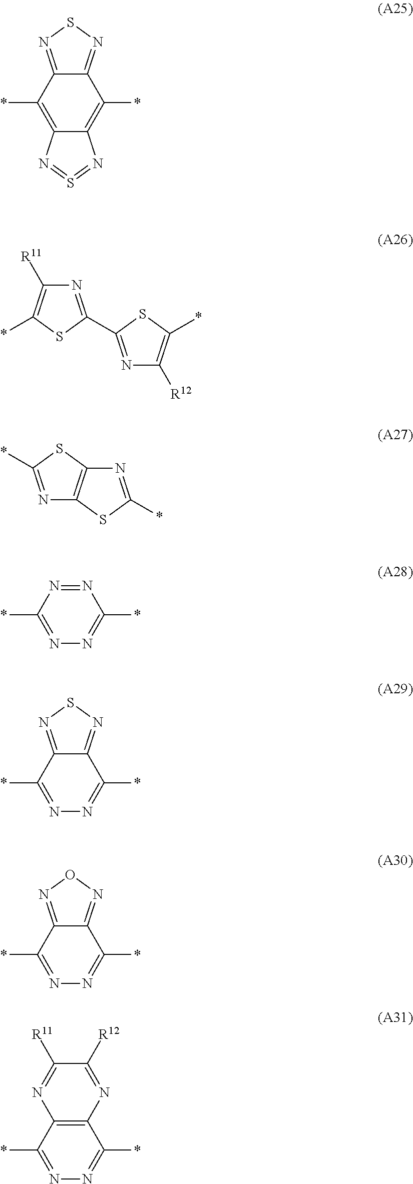

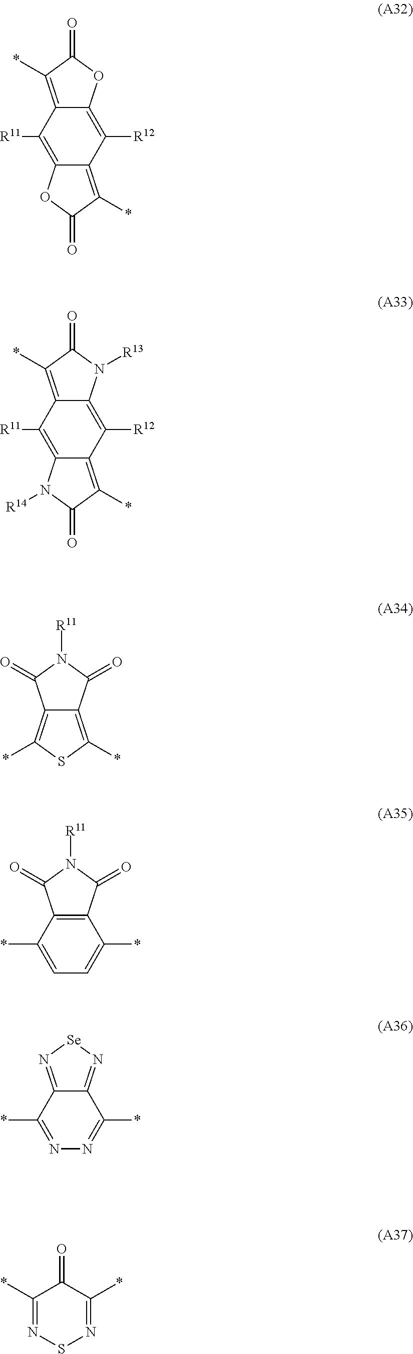

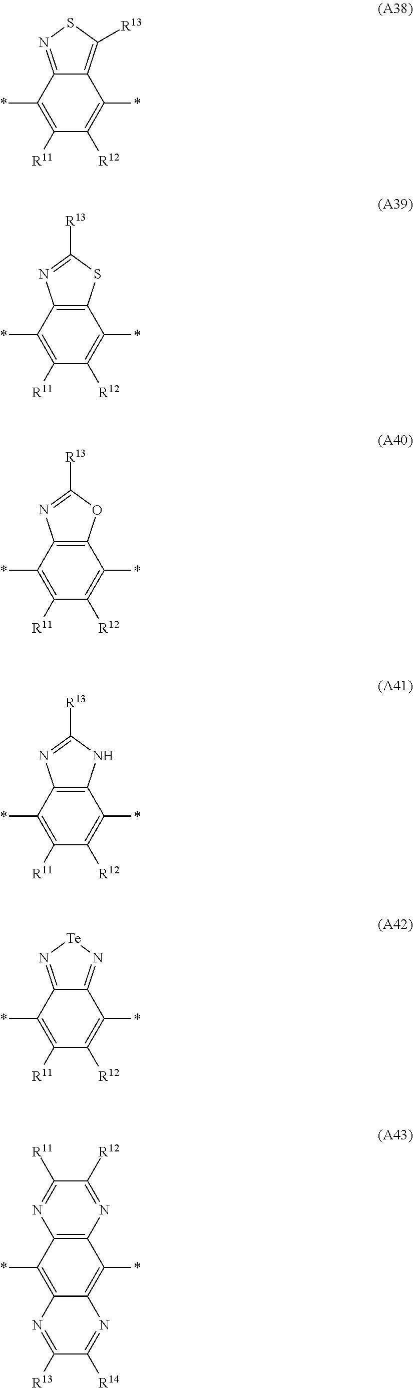

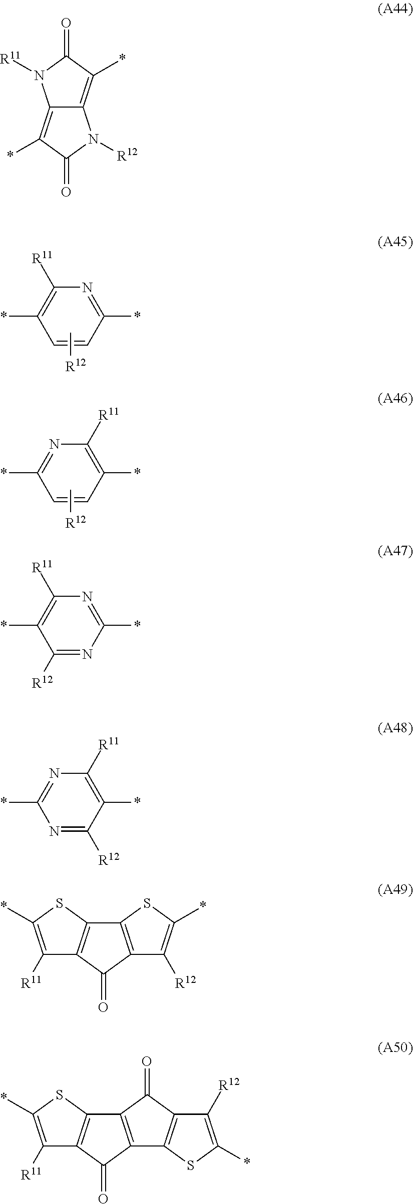

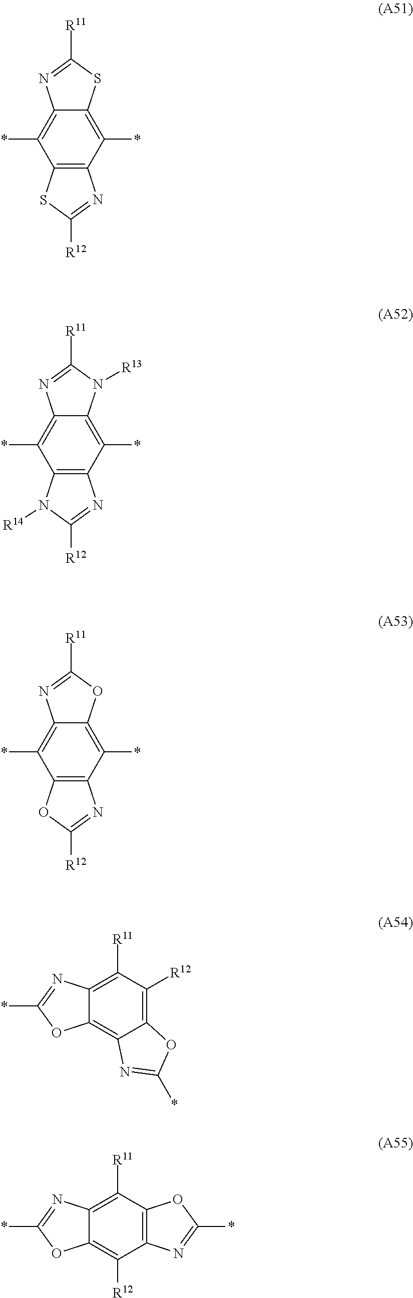

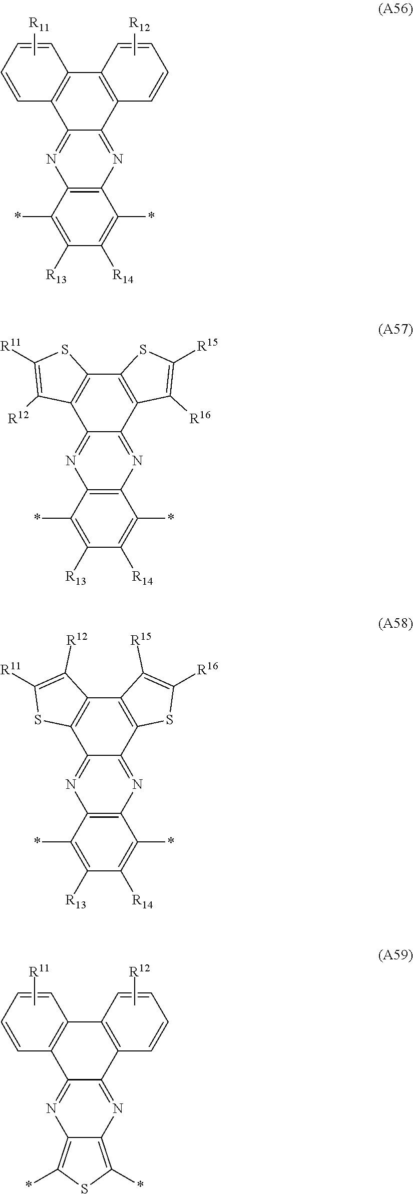

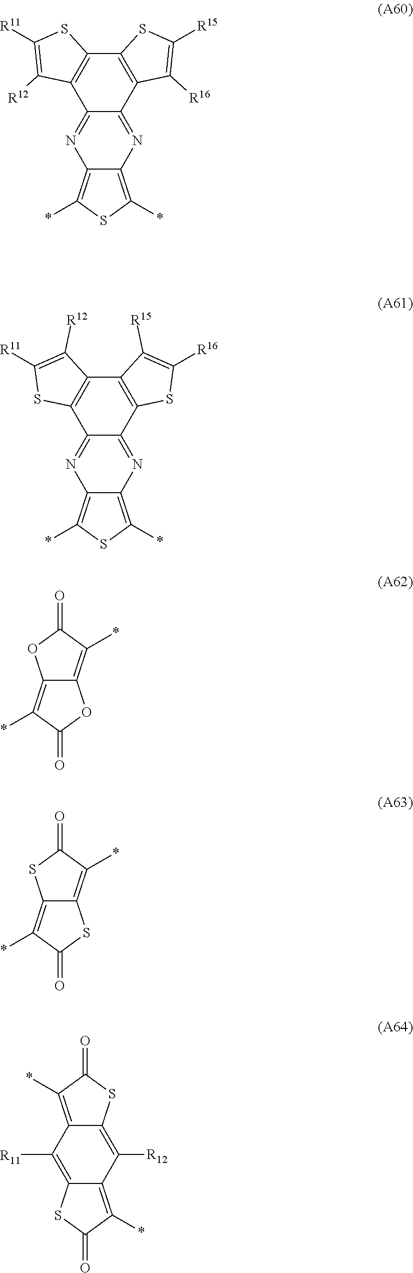

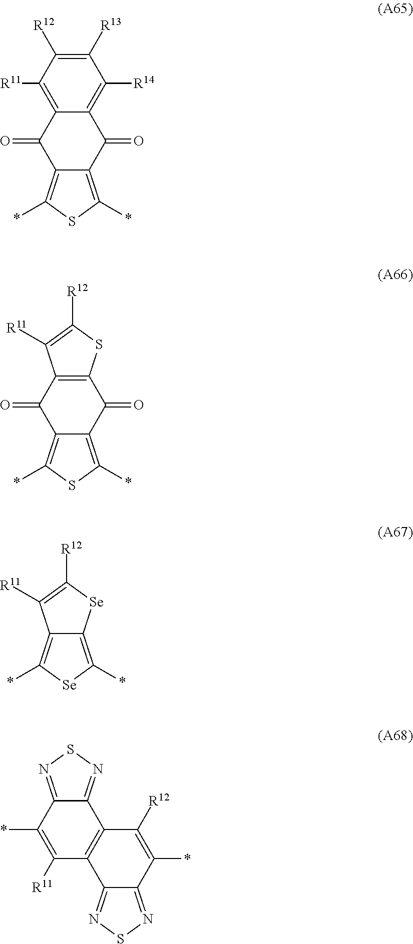

wherein one of X¹¹ and X¹² is S and the other is Se, and R¹¹, R¹², R¹³, R¹⁴ and R¹⁵ independently of each other denote H or have one of the meanings of R¹ as defined above and below.

In compounds of formula I and II wherein one or more of Ar¹⁻¹¹ denote U, all of the groups U that are present in the compounds of formula I and II are preferably not linked directly to each other.

In compounds of formula I and II wherein one or more of Ar¹⁻¹¹ denote U, all of the groups U that are present in these compounds may have the same structure or may have different structure. Preferably all of the groups U present in the compounds of formula I and II have the same structure.

Especially preferred compounds of formula I are selected from the following subformulae

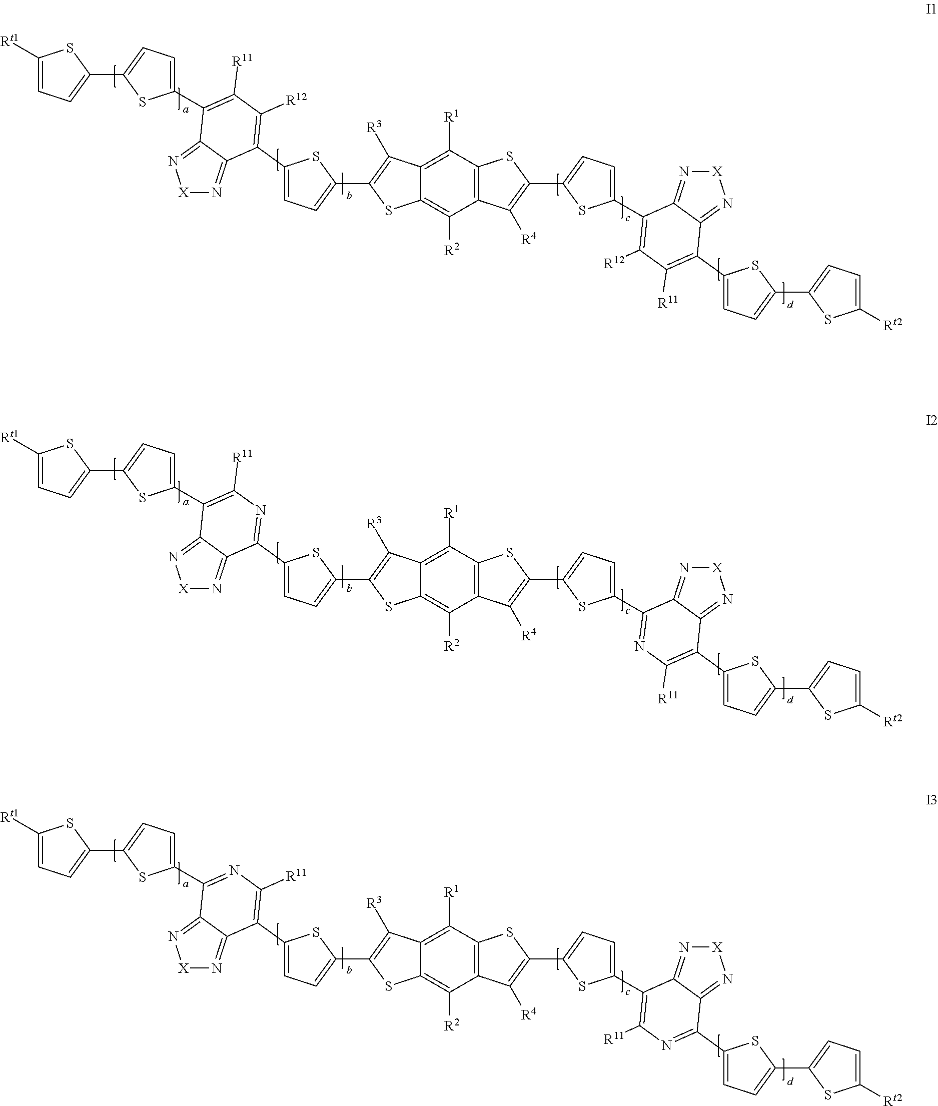

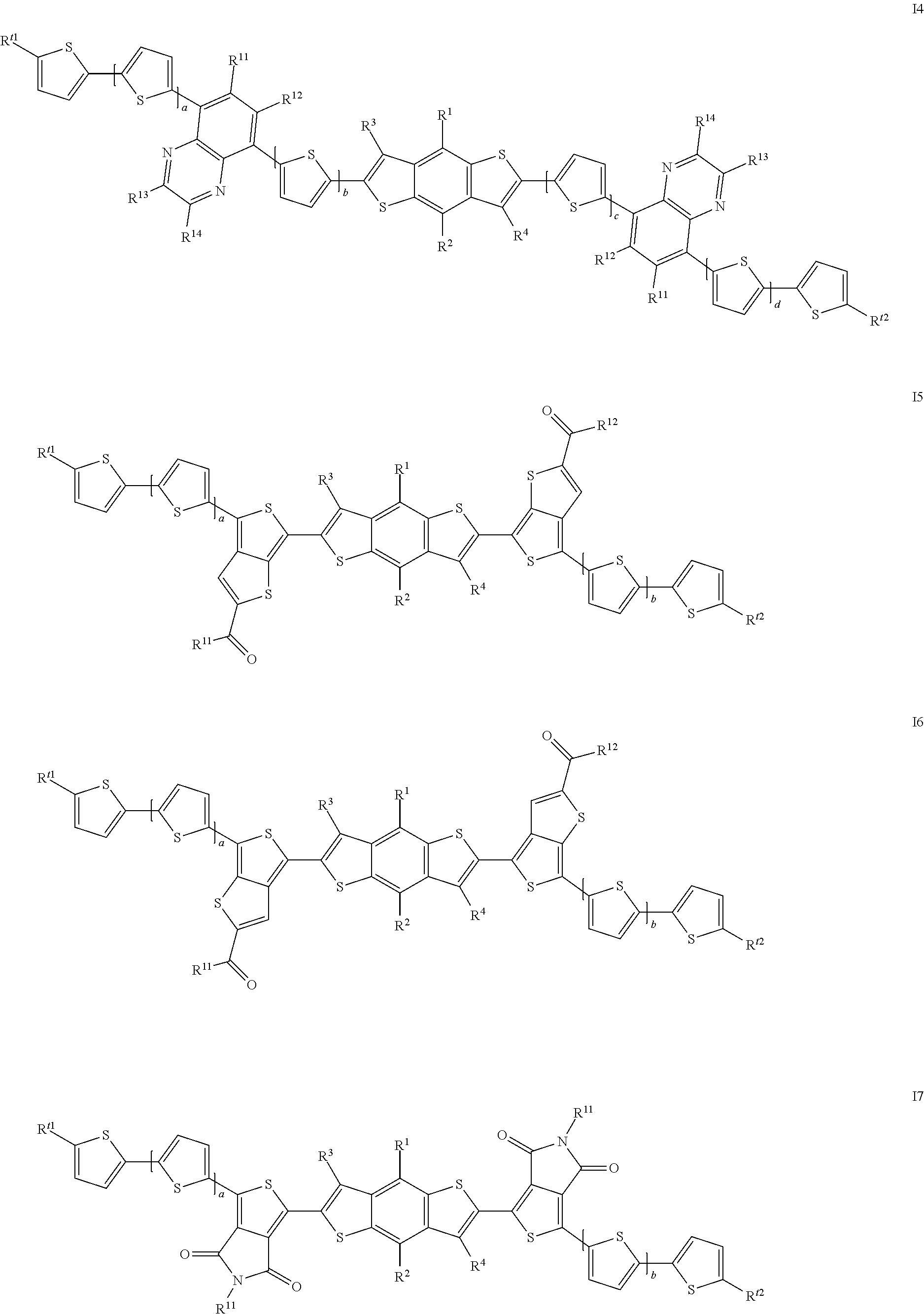

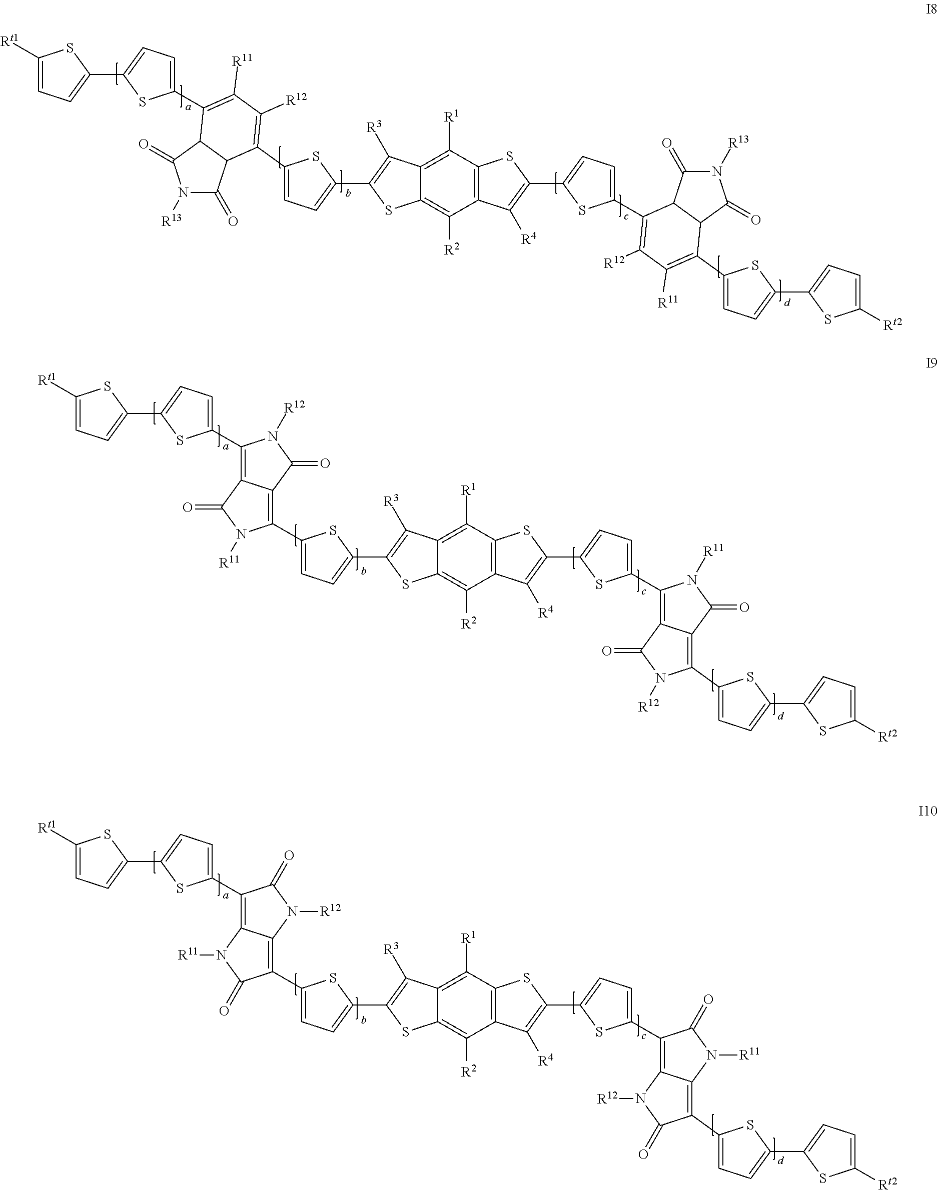

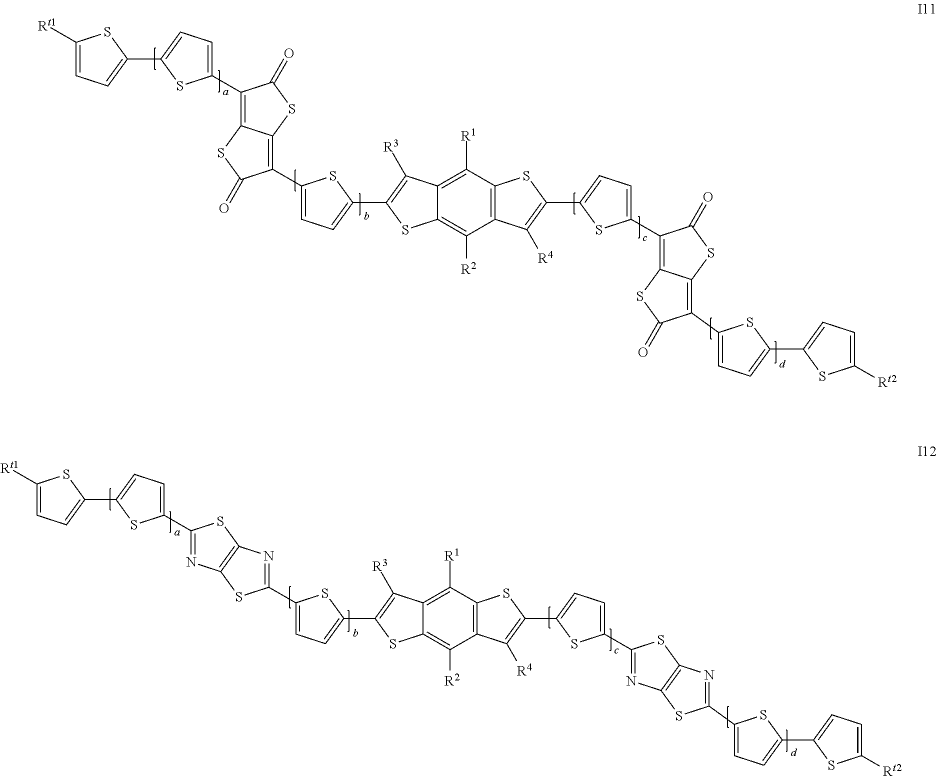

wherein R¹⁻⁴, R^{t1}, R^{t2} have the meanings given in formula I,
X denotes NR, O, S or Se, with R being as defined in formula I,
R¹¹⁻¹⁴ have one of the meanings given for R¹, and preferably denote H or alkoxy with 1 to 20 C atoms,
a, b, c and d are 0 or 1, with a+b+c+d≧0, and preferably a=b=c=d=1.

Further preferred are compounds of formula I and II and their subformulae that are selected from the following list of preferred embodiments:
one or more of Ar¹⁻⁸ denote U,
R³ and R⁴ are H,
R³ and R⁴ are H, and R¹ and R² are different from H,
R¹ and R² are H, and R³ and R⁴ are different from H,
R¹ and/or R² are independently of each other selected from the group consisting of primary alkyl or sulfanylalkyl with 1 to 30 C atoms, secondary alkyl or sulfanylalkyl with 3 to 30 C atoms, and tertiary alkyl or sulfanylalkyl with 4 to 30 C atoms, wherein in all these groups one or more H atoms are optionally replaced by F,
R¹ and/or R² denote independently of each other F, Cl, Br, I, CN, —CF₃, —CF₂—R⁹, —C(O)—R⁹, —C(O)—O—R⁹, —O—C(O)—R⁹, —SO₂—R⁹, wherein R⁹ is straight-chain, branched or cyclic alkyl with 1 to 30 C atoms, in which one or more C atoms are optionally replaced by —O—, —S—, —C(O)—, —C(S)—, —NR⁰R⁰⁰—, —CHR⁰═CR⁰⁰— or —C≡C— such that O- and/or S-atoms are not directly linked to each other, and in which one or more H atoms are optionally replaced by F, CI or CN,
R³ and/or R⁴ are independently of each other selected from the group consisting of primary alkyl with 1 to 30 C atoms, secondary alkyl with 3 to 30 C atoms, and tertiary alkyl with 4 to 30 C atoms, wherein in all these groups one or more H atoms are optionally replaced by F,
R³ and/or R⁴ are independently of each other selected from the group consisting of primary alkoxy or sulfanylalkyl with 1 to 30 C atoms, secondary alkoxy or sulfanylalkyl with 3 to 30 C atoms, and tertiary alkoxy or sulfanylalkyl with 4 to 30 C atoms, wherein in all these groups one or more H atoms are optionally replaced by F,
R¹ and/or R² denote independently of each other F, Cl, Br, I, CN, —CF₃, —CF₂—R⁹, —C(O)—R⁹, —C(O)—O—R⁹, —O—C(O)—R⁹, —SO₂—R⁹, wherein R⁹ is straight-chain, branched or cyclic alkyl with 1 to 30 C atoms, in which one or more C atoms are optionally replaced by —O—, —S—, —C(O)—, —C(S)—, —NR⁰R⁰⁰—, —CHR⁰═CR⁰⁰— or —C≡C— such that O- and/or S-atoms are not directly linked to each other, and in which one or more H atoms are optionally replaced by F, CI or CN,
R⁰ and R⁰⁰ are selected from H or C₁-C₁₀-alkyl,
R⁵ and R⁶ are, preferably independently of each other, selected from the group consisting of Cl, Br, I, O-tosylate, O-triflate, O-mesylate, O-nonaflate, —B(OZ²)₂, —ZnX′ and —Sn(Z⁴)₃, wherein Z², Z⁴ and X′ are as defined above.

The compounds of formula I and II can be synthesized according to or in analogy to methods that are known to the skilled person and are described in the literature. Other methods of preparation can be taken from the examples. Preferred and suitable synthesis methods are further described in the reaction schemes shown below, wherein R¹⁻⁴ and Ar¹⁻⁸ are as defined in formula I.

The generic preparation of the benzo[1,2-b:4,5-b′]dithiophene core has been described for example in WO 2011/085004 A2, WO 2011/131280 A1, and U.S. Pat. No. 7,524,922 B2.

The generic synthesis scheme for the symmetric benzo[1,2-b:4,5-b′]dithiophene based organic semiconductors is shown in Schemes 1 and 2.

As shown in Scheme 1, the generic synthesis of symmetric benzo[1,2-b:4,5-b′]dithiophene core organic semiconductors can be carried out via a sequential synthesis strategy where Ar₅-Ar₆-Ar₇-Ar₈-R^{t2} is identical to Ar₄-Ar₃-Ar₂-Ar₁-R^{t1}

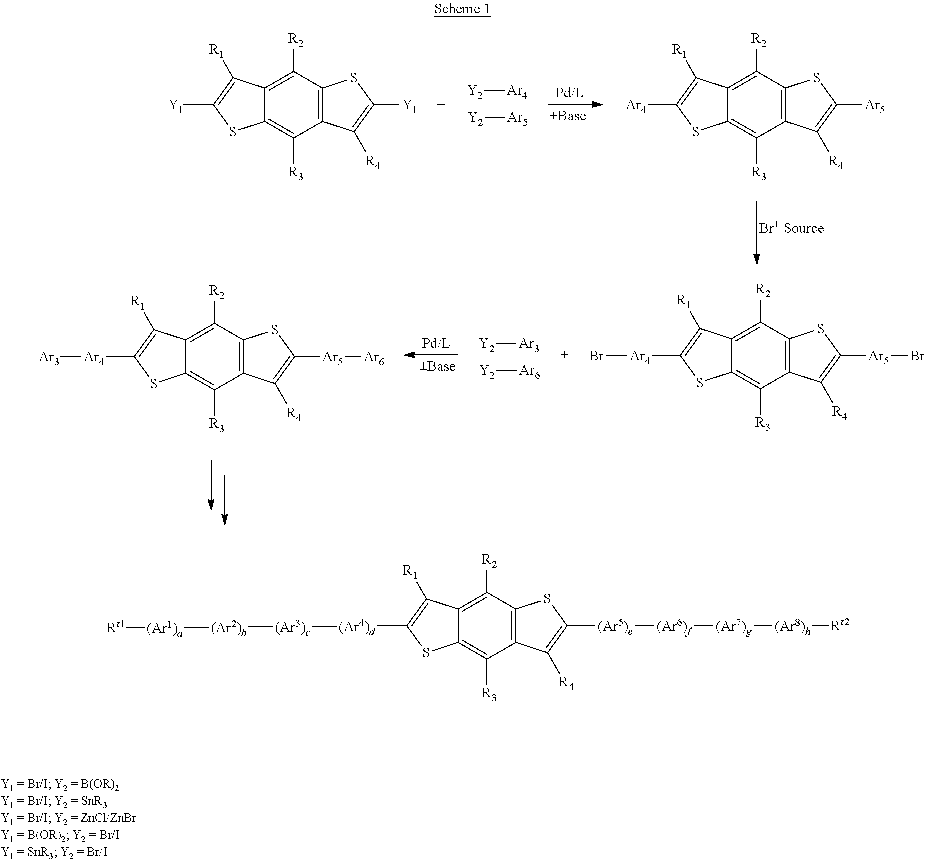

Alternatively the benzo[1,2-b:4,5-b′]dithiophene based organic semiconductor can be obtain via a convergent synthesis strategy as shown in Scheme 2, where Y₂-Ar₅-Ar₆-Ar₇-Ar₈-R^{t2} is identical to Y₂-Ar₄-Ar₃-Ar₂-Ar₁-R^{t1} and Ar₅-Ar₆-Ar₇-Ar₈-R^{t2} is identical to Ar₄-Ar₃-Ar₂-Ar₁-R^{t1}.

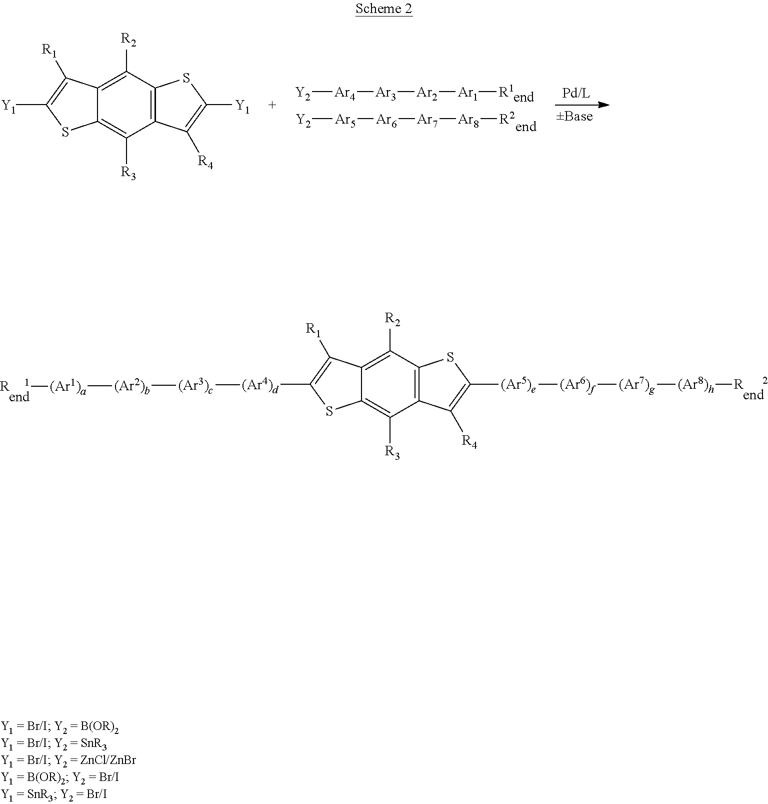

The generic synthesis scheme for the asymmetric benzo[1,2-b:4,5-b′]dithiophene based organic semiconductors is shown in Schemes 3 and 4.

As shown in Scheme 3, the generic synthesis of asymmetric benzo[1,2-b:4,5-b′]dithiophene core organic semiconductors can be carried out via a sequential synthesis strategy.

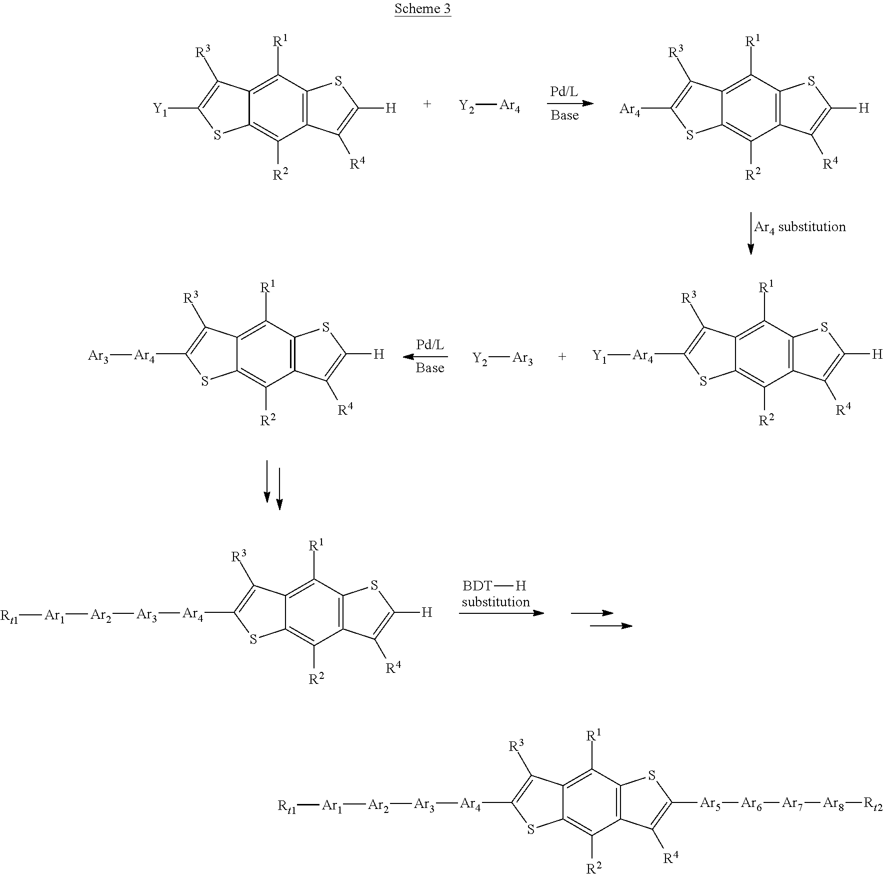

wherein Y₁ and Y₂ are as defined in Scheme 2.

Alternatively the asymmetric benzo[1,2-b:4,5-b′]dithiophene based organic semiconductor can be obtain via a convergent synthesis strategy as shown in Scheme 4.

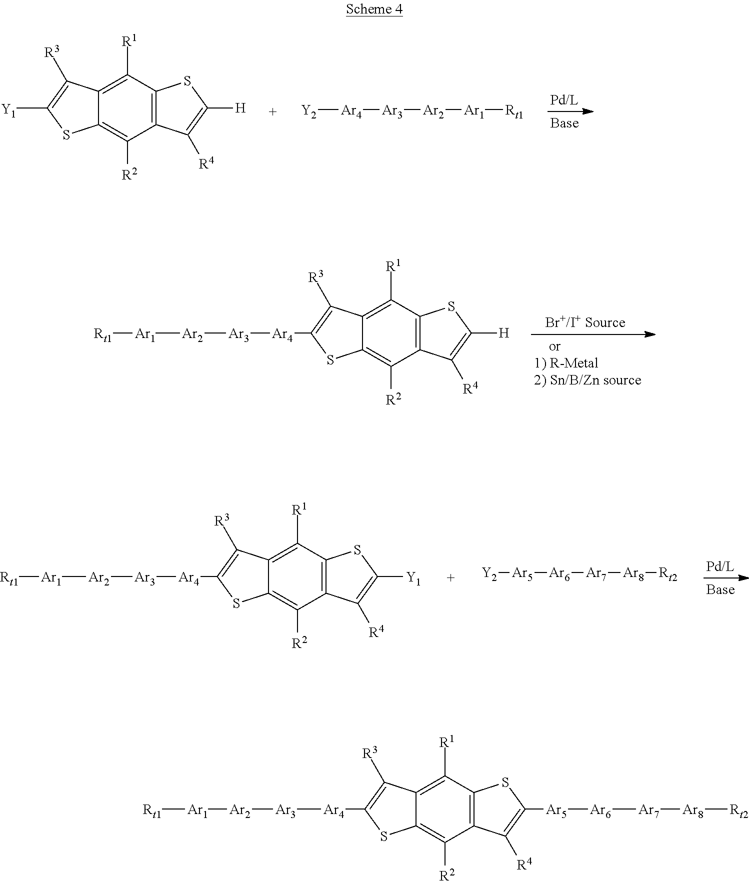

wherein Y₁ and Y₂ are as defined in Scheme 2.

The generic synthesis for the bis-benzo[1,2-b:4,5-b′]dithiophene based organic semiconductors is shown in Scheme 5.

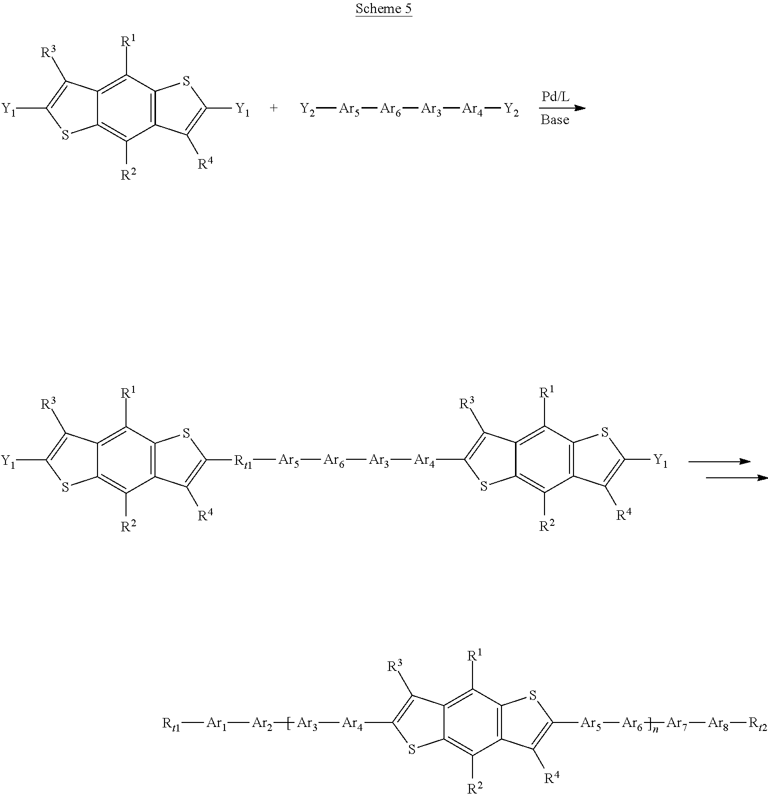

wherein Y₁ and Y₂ are as defined in Scheme 2.

Further substitution can be added to the benzo[1,2-b:4,5-b′]dithiophene core at the R^{t12} substitution after the benzo[1,2-b:4,5-b′]dithiophene core organic semiconductors have been prepared, as shown in Scheme 6.

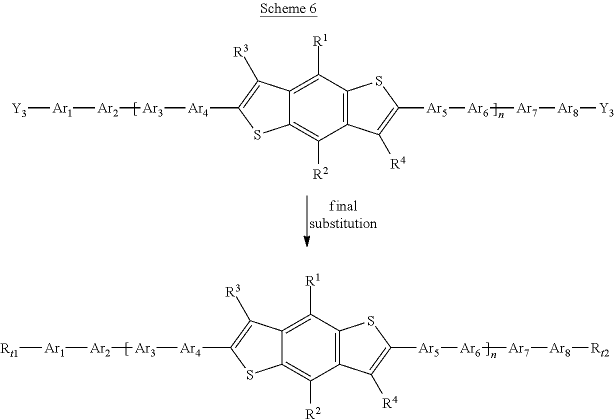

The novel methods of preparing compounds as described above and below and the intermediates used therein are further aspects of the present invention.

Preferred aryl-aryl coupling methods used in the processes described above are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. For example, when using Yamamoto coupling, compounds of formula II having two reactive halide groups are preferably used. When using Suzuki coupling, compounds of formula II having two reactive boronic acid or boronic acid ester groups or two reactive halide groups are preferably used. When using Stille coupling, compounds of formula II having two reactive stannane groups or two reactive halide groups are preferably used. When using Negishi coupling, compounds of formula II having two reactive organozinc groups or two reactive halide groups are preferably used.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(OAc)₂. Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine or tri(tert-butyl)phosphine. Suzuki coupling is performed in the presence of a base, for example sodium carbonate, potassium carbonate, lithium hydroxide, potassium phosphate or an organic base such as tetraethylammonium carbonate or tetraethylammonium hydroxide. Yamamoto coupling employs a Ni(0) complex, for example bis(1,5-cyclooctadienyl) nickel(0).

The invention further relates to a formulation comprising one or more compounds of formula I and one or more solvents, preferably selected from organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetramethyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylansiole, 3-methylanisole, 4-fluoro-3-methylanisole, 2-fluorobenzonitrile, 4-fluoroveratrol, 2,6-dimethylanisole, 3-fluorobenzonitrile, 2,5-dimethylanisole, 2,4-dimethylanisole, benzonitrile, 3,5-dimethylanisole, N,N-dimethylaniline, ethyl benzoate, 1-fluoro-3,5-dimethoxybenzene, 1-methylnaphthalene, N-methylpyrrolidinone, 3-fluorobenzotrifluoride, benzotrifluoride, dioxane, trifluoromethoxybenzene, 4-fluorobenzotrifluoride, 3-fluoropyridine, toluene, 2-fluorotoluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluorobenzene, 1-chloro-2,5-difluorobenzene, 4-chlorofluorobenzene, chlorobenzene, o-dichlorobenzene, 2-chlorofluorobenzene, p-xylene, m-xylene, o-xylene or mixtures of o-, m-, and p-isomers. Solvents with relatively low polarity are generally preferred. For inkjet printing solvents with high boiling temperatures and solvent mixtures are preferred. For spin coating alkylated benzenes like xylene and toluene are preferred.

The invention further relates to an organic semiconducting formulation comprising one or more compounds of formula I, one or more organic binders, or precursors thereof, preferably having a permittivity ∈ at 1,000 Hz of 3.3 or less, and optionally one or more solvents.

Combining specified soluble compounds of formula I, especially compounds of the preferred formulae as described above and below, with an organic binder resin (hereinafter also referred to as “the binder”) results in little or no reduction in charge mobility of the compounds of formula I, even an increase in some instances. For instance, the compounds of formula I may be dissolved in a binder resin (for example poly(α-methylstyrene) and deposited (for example by spin coating), to form an organic semiconducting layer yielding a high charge mobility. Moreover, a semiconducting layer formed thereby exhibits excellent film forming characteristics and is particularly stable.

If an organic semiconducting layer formulation of high mobility is obtained by combining a compound of formula I with a binder, the resulting formulation leads to several advantages. For example, since the compounds of formula I are soluble they may be deposited in a liquid form, for example from solution. With the additional use of the binder the formulation can be coated onto a large area in a highly uniform manner. Furthermore, when a binder is used in the formulation it is possible to control the properties of the formulation to adjust to printing processes, for example viscosity, solid content, surface tension. Whilst not wishing to be bound by any particular theory it is also anticipated that the use of a binder in the formulation fills in volume between crystalline grains otherwise being void, making the organic semiconducting layer less sensitive to air and moisture. For example, layers formed according to the process of the present invention show very good stability in OFET devices in air.

The invention also provides an organic semiconducting layer which comprises the organic semiconducting layer formulation.

The invention further provides a process for preparing an organic semiconducting layer, said process comprising the following steps:
(i) depositing on a substrate a liquid layer of a formulation comprising one or more compounds of formula I as described above and below, one or more organic binder resins or precursors thereof, and optionally one or more solvents,
(ii) forming from the liquid layer a solid layer which is the organic semiconducting layer,
(iii) optionally removing the layer from the substrate.

The process is described in more detail below.

The invention additionally provides an electronic device comprising the said organic semiconducting layer. The electronic device may include, without limitation, an organic field effect transistor (OFET), organic light emitting diode (OLED), photodetector, sensor, logic circuit, memory element, capacitor or photovoltaic (PV) cell. For example, the active semiconductor channel between the drain and source in an OFET may comprise the layer of the invention. As another example, a charge (hole or electron) injection or transport layer in an OLED device may comprise the layer of the invention.

The formulations according to the present invention and layers formed therefrom have particular utility in OFETs especially in relation to the preferred embodiments described herein.

The semiconducting compound of formula I preferably has a charge carrier mobility, μ, of more than 0.001 cm²V⁻¹s⁻¹, very preferably of more than 0.01 cm²V⁻¹s⁻¹, especially preferably of more than 0.1 cm²V⁻¹s⁻¹ and most preferably of more than 0.5 cm²V⁻¹s⁻¹.

The binder, which is typically a polymer, may comprise either an insulating binder or a semiconducting binder, or mixtures thereof may be referred to herein as the organic binder, the polymeric binder or simply the binder.

Preferred binders according to the present invention are materials of low permittivity, that is, those having a permittivity ∈ of 3.3 or less. The organic binder preferably has a permittivity ∈ of 3.0 or less, more preferably 2.9 or less. Preferably the organic binder has a permittivity ∈ at of 1.7 or more. It is especially preferred that the permittivity of the binder is in the range from 2.0 to 2.9. Whilst not wishing to be bound by any particular theory it is believed that the use of binders with a permittivity ∈ of greater than 3.3, may lead to a reduction in the OSC layer mobility in an electronic device, for example an OFET. In addition, high permittivity binders could also result in increased current hysteresis of the device, which is undesirable.

An example of a suitable organic binder is polystyrene. Further examples of suitable binders are disclosed for example in US 2007/0102696 A1. Especially suitable and preferred binders are described in the following.

In one type of preferred embodiment, the organic binder is one in which at least 95%, more preferably at least 98% and especially all of the atoms consist of hydrogen, fluorine and carbon atoms.

It is preferred that the binder normally contains conjugated bonds, especially conjugated double bonds and/or aromatic rings.

The binder should preferably be capable of forming a film, more preferably a flexible film. Polymers of styrene and α-methyl styrene, for example copolymers including styrene, α-methylstyrene and butadiene may suitably be used.

Binders of low permittivity of use in the present invention have few permanent dipoles which could otherwise lead to random fluctuations in molecular site energies. The permittivity ∈ (dielectric constant) can be determined by the ASTM D150 test method.

The permittivity values given above and below, unless stated otherwise, refer to 1,000 Hz and 20° C.

It is also preferred that in the present invention binders are used which have solubility parameters with low polar and hydrogen bonding contributions as materials of this type have low permanent dipoles. A preferred range for the solubility parameters (‘Hansen parameter’) of a binder for use in accordance with the present invention is provided in Table 1 below.

TABLE 1

| | Hansen parameter | | |
|---|---|---|---|
| | δ_{d} MPa^{1/2} | δₚ MPa^{1/2} | δₕ MPa^{1/2} |
| Preferred range | 14.5+ | 0-10 | 0-14 |
| More preferred range | 16+ | 0-9 | 0-12 |
| Most preferred range | 17+ | 0-8 | 0-10 |

The three dimensional solubility parameters listed above include: dispersive (δ_{d}), polar (δₚ) and hydrogen bonding (δₕ) components (C. M. Hansen, Ind. Eng. and Chem., Prod. Res. and Devl., 9, No 3, p 282., 1970). These parameters may be determined empirically or calculated from known molar group contributions as described in Handbook of Solubility Parameters and Other Cohesion Parameters ed. A. F. M. Barton, CRC Press, 1991. The solubility parameters of many known polymers are also listed in this publication.

It is desirable that the permittivity of the binder has little dependence on frequency. This is typical of non-polar materials. Polymers and/or copolymers can be chosen as the binder by the permittivity of their substituent groups. A list of suitable and preferred low polarity binders is given (without limiting to these examples) in Table 2:

TABLE 2

| | typical low |
|---|---|
| | frequency |
| Binder | permittivity (ε) |
| polystyrene | 2.5 |
| poly(α-methylstyrene) | 2.6 |
| poly(α-vinylnaphtalene) | 2.6 |
| poly(vinyltoluene) | 2.6 |
| polyethylene | 2.2-2.3 |
| cis-polybutadiene | 2.0 |
| polypropylene | 2.2 |
| poly(4-methyl-1-pentene) | 2.1 |
| poly(4-methylstyrene) | 2.7 |
| poly(chorotrifluoroethylene) | 2.3-2.8 |
| poly(2-methyl-1,3-butadiene) | 2.4 |
| poly(p-xylylene) | 2.6 |
| poly(α-α-α′-α′ tetrafluoro-p-xylylene) | 2.4 |
| poly[1,1-(2-methyl propane)bis(4-phenyl)carbonate] | 2.3 |
| poly(cyclohexyl methacrylate) | 2.5 |
| poly(chlorostyrene) | 2.6 |
| poly(2,6-dimethyl-1,4-phenylene ether) | 2.6 |
| polyisobutylene | 2.2 |
| poly(vinyl cyclohexane) | 2.2 |
| poly(vinylcinnamate) | 2.9 |
| poly(4-vinylbiphenyl) | 2.7 |

Further preferred binders are poly(1,3-butadiene) and polyphenylene.

Especially preferred are formulations wherein the binder is selected from poly-α-methyl styrene, polystyrene and polytriarylamine or any copolymers of these, and the solvent is selected from xylene(s), toluene, tetralin and cyclohexanone.

Copolymers containing the repeat units of the above polymers are also suitable as binders. Copolymers offer the possibility of improving compatibility with the compounds of formula I, modifying the morphology and/or the glass transition temperature of the final layer composition. It will be appreciated that in the above table certain materials are insoluble in commonly used solvents for preparing the layer. In these cases analogues can be used as copolymers. Some examples of copolymers are given in Table 3 (without limiting to these examples). Both random or block copolymers can be used. It is also possible to add more polar monomer components as long as the overall composition remains low in polarity.

TABLE 3

| | typical low |
|---|---|
| | frequency |
| Binder | permittivity (ε) |
| poly(ethylene/tetrafluoroethylene) | 2.6 |
| poly(ethylene/chlorotrifluoroethylene) | 2.3 |
| fluorinated ethylene/propylene copolymer | 2-2.5 |
| polystyrene-co-α-methylstyrene | 2.5-2.6 |
| ethylene/ethyl acrylate copolymer | 2.8 |
| poly(styrene/10%butadiene) | 2.6 |
| poly(styrene/15%butadiene) | 2.6 |
| poly(styrene/2,4 dimethylstyrene) | 2.5 |
| Topas ™ (all grades) | 2.2-2.3 |

Other copolymers may include: branched or non-branched polystyrene-block-polybutadiene, polystyrene-block(polyethylene-ran-butylene)-block-polystyrene, polystyrene-block-polybutadiene-block-polystyrene, polystyrene-(ethylene-propylene)-diblock-copolymers (e.g. KRATON®-G1701 E, Shell), poly(propylene-co-ethylene) and poly(styrene-co-methylmethacrylate).

Preferred insulating binders for use in the organic semiconductor layer formulation according to the present invention are poly(α-methylstyrene), polyvinylcinnamate, poly(4-vinylbiphenyl), poly(4-methylstyrene), and Topas™ 8007 (linear olefin, cyclo-olefin(norbornene) copolymer available from Ticona, Germany). Most preferred insulating binders are poly(α-methylstyrene), polyvinylcinnamate and poly(4-vinylbiphenyl).

The binder can also be selected from crosslinkable binders, like e.g. acrylates, epoxies, vinylethers, thiolenes etc., preferably having a sufficiently low permittivity, very preferably of 3.3 or less. The binder can also be mesogenic or liquid crystalline.

As mentioned above the organic binder may itself be a semiconductor, in which case it will be referred to herein as a semiconducting binder. The semiconducting binder is still preferably a binder of low permittivity as herein defined. Semiconducting binders for use in the present invention preferably have a number average molecular weight (Mₙ) of at least 1500-2000, more preferably at least 3000, even more preferably at least 4000 and most preferably at least 5000. The semiconducting binder preferably has a charge carrier mobility, pI, of at least 10⁻⁵ cm²V⁻¹s⁻¹, more preferably at least 10⁻⁴ cm²V⁻¹s⁻¹.

A preferred class of semiconducting binder is a polymer as disclosed in U.S. Pat. No. 6,630,566, preferably an oligomer or polymer having repeat units of formula 1:

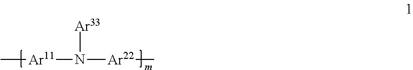

wherein
Ar¹¹, Ar²² and Ar³³ which may be the same or different, denote, independently if in different repeat units, an optionally substituted aromatic group that is mononuclear or polynuclear, and
m is an integer ≧1, preferably ≧6, preferably ≧10, more preferably ≧15 and most preferably ≧20.

In the context of Ar¹¹, Ar²² and Ar³³, a mononuclear aromatic group has only one aromatic ring, for example phenyl or phenylene. A polynuclear aromatic group has two or more aromatic rings which may be fused (for example napthyl or naphthylene), individually covalently linked (for example biphenyl) and/or a combination of both fused and individually linked aromatic rings. Preferably each Ar¹¹, Ar²² and Ar³³ is an aromatic group which is substantially conjugated over substantially the whole group.

Further preferred classes of semiconducting binders are those containing substantially conjugated repeat units. The semiconducting binder polymer may be a homopolymer or copolymer (including a block-copolymer) of the general formula 2:

A_{(c)}B_{(d)} . . . Z_{(z)} 2

wherein A, B, . . . , Z each represent a monomer unit and (c), (d), . . . (z) each represent the mole fraction of the respective monomer unit in the polymer, that is each (c), (d), . . . (z) is a value from 0 to 1 and the total of (c)+(d)+ . . . +(z)=1.

Examples of suitable and preferred monomer units A, B, . . . Z include units of formula 1 above and of formulae 3 to 8 given below (wherein m is as defined in formula 1:

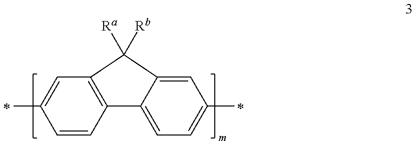

wherein
R^{a} and R^{b} are independently of each other selected from H, F, CN, NO₂, —N(R^{c})(R^{d}) or optionally substituted alkyl, alkoxy, thioalkyl, acyl, aryl,
R^{c} and R^{d} are independently or each other selected from H, optionally substituted alkyl, aryl, alkoxy or polyalkoxy or other substituents,
   and wherein the asterisk (*) is any terminal or end capping group including H, and the alkyl and aryl groups are optionally fluorinated;

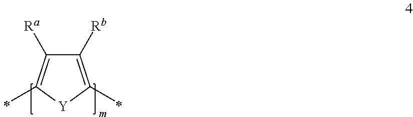

wherein
Y is Se, Te, O, S or —N(R^{e}), preferably O, S or —N(R^{e})—,
R^{e} is H, optionally substituted alkyl or aryl,
R^{a} and R^{b} are as defined in formula 3;

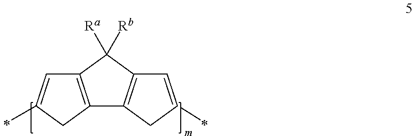

wherein R^{a}, R^{b} and Y are as defined in formulae 3 and 4;

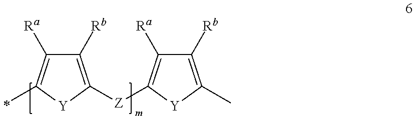

wherein R^{a}, R^{b} and Y are as defined in formulae 3 and 4,
Z is —C(T¹)═C(T²)—, —C≡C—, —N(R^{f})—, —N═N—, (R^{f})═N—, —N═C(R^{f})—,
T¹ and T² independently of each other denote H, Cl, F, —CN or lower alkyl with 1 to 8 C atoms,
R^{f} is H or optionally substituted alkyl or aryl;

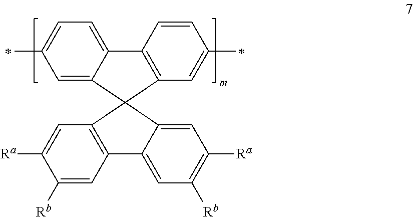

wherein R^{a} and R^{b} are as defined in formula 3;

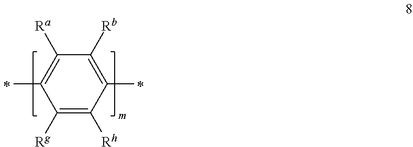

wherein R^{a}, R^{b}, R^{g} and R^{h} independently of each other have one of the meanings of R^{a} and R^{b} in formula 3.

In the case of the polymeric formulae described herein, such as formulae 1 to 8, the polymers may be terminated by any terminal group, that is any end-capping or leaving group, including H.

In the case of a block-copolymer, each monomer A, B, . . . Z may be a conjugated oligomer or polymer comprising a number, for example 2 to 50, of the units of formulae 3-8. The semiconducting binder preferably includes: arylamine, fluorene, thiophene, spiro bifluorene and/or optionally substituted aryl (for example phenylene) groups, more preferably arylamine, most preferably triarylamine groups. The aforementioned groups may be linked by further conjugating groups, for example vinylene.

In addition, it is preferred that the semiconducting binder comprises a polymer (either a homo-polymer or copolymer, including block-copolymer) containing one or more of the aforementioned arylamine, fluorene, thiophene and/or optionally substituted aryl groups. A preferred semiconducting binder comprises a homo-polymer or copolymer (including block-copolymer) containing arylamine (preferably triarylamine) and/or fluorene units. Another preferred semiconducting binder comprises a homo-polymer or co-polymer (including block-copolymer) containing fluorene and/or thiophene units.

The semiconducting binder may also contain carbazole or stilbene repeat units. For example, polyvinylcarbazole, polystilbene or their copolymers may be used. The semiconducting binder may optionally contain DBBDT segments (for example repeat units as described for formula 1 above) to improve compatibility with the soluble compounds of formula.

Very preferred semiconducting binders for use in the organic semiconductor formulation according to the present invention are poly(9-vinylcarbazole) and PTAA1, a polytriarylamine of the following formula

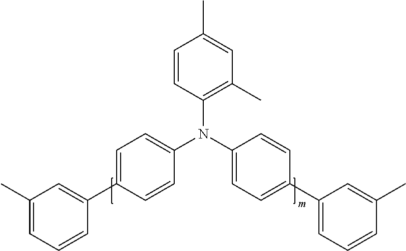

wherein m is as defined in formula 1.

For application of the semiconducting layer in p-channel FETs, it is desirable that the semiconducting binder should have a higher ionisation potential than the semiconducting compound of formula I, otherwise the binder may form hole traps. In n-channel materials the semiconducting binder should have lower electron affinity than the n-type semiconductor to avoid electron trapping.

The formulation according to the present invention may be prepared by a process which comprises:
(i) first mixing a compound of formula I and an organic binder or a precursor thereof. Preferably the mixing comprises mixing the two components together in a solvent or solvent mixture,
(ii) applying the solvent(s) containing the compound of formula I and the organic binder to a substrate; and optionally evaporating the solvent(s) to form a solid organic semiconducting layer according to the present invention,
(iii) and optionally removing the solid layer from the substrate or the substrate from the solid layer.

In step (i) the solvent may be a single solvent or the compound of formula I and the organic binder may each be dissolved in a separate solvent followed by mixing the two resultant solutions to mix the compounds.

The binder may be formed in situ by mixing or dissolving a compound of formula I in a precursor of a binder, for example a liquid monomer, oligomer or crosslinkable polymer, optionally in the presence of a solvent, and depositing the mixture or solution, for example by dipping, spraying, painting or printing it, on a substrate to form a liquid layer and then curing the liquid monomer, oligomer or crosslinkable polymer, for example by exposure to radiation, heat or electron beams, to produce a solid layer. If a preformed binder is used it may be dissolved together with the compound of formula I in a suitable solvent, and the solution deposited for example by dipping, spraying, painting or printing it on a substrate to form a liquid layer and then removing the solvent to leave a solid layer. It will be appreciated that solvents are chosen which are able to dissolve both the binder and the compound of formula I, and which upon evaporation from the solution blend give a coherent defect free layer.

Suitable solvents for the binder or the compound of formula I can be determined by preparing a contour diagram for the material as described in ASTM Method D 3132 at the concentration at which the mixture will be employed. The material is added to a wide variety of solvents as described in the ASTM method.

It will also be appreciated that in accordance with the present invention the formulation may also comprise two or more compounds of formula I and/or two or more binders or binder precursors, and that the process for preparing the formulation may be applied to such formulations.

Examples of suitable and preferred organic solvents include, without limitation, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, tetralin, decalin, indane and/or mixtures thereof.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38(496), 296”. Solvent blends may also be used and can be identified as described in “Solvents, W. H. Ellis, Federation of Societies for Coatings Technology, p 9-10, 1986”. Such a procedure may lead to a blend of ‘non’ solvents that will dissolve both the binder and the compound of formula I, although it is desirable to have at least one true solvent in a blend.

Especially preferred solvents for use in the formulation according to the present invention, with insulating or semiconducting binders and mixtures thereof, are xylene(s), toluene, tetralin and o-dichlorobenzene.

The proportions of binder to the compound of formula I in the formulation or layer according to the present invention are typically 20:1 to 1:20 by weight, preferably 10:1 to 1:10 more preferably 5:1 to 1:5, still more preferably 3:1 to 1:3 further preferably 2:1 to 1:2 and especially 1:1. Surprisingly and beneficially, dilution of the compound of formula I in the binder has been found to have little or no detrimental effect on the charge mobility, in contrast to what would have been expected from the prior art.

In accordance with the present invention it has further been found that the level of the solids content in the organic semiconducting layer formulation is also a factor in achieving improved mobility values for electronic devices such as OFETs. The solids content of the formulation is commonly expressed as follows:

$$Solids content ( \% ) = \frac{a + b}{a + b + c} \times 100$$

wherein a=mass of compound of formula I, b=mass of binder and c=mass of solvent.

The solids content of the formulation is preferably 0.1 to 10% by weight, more preferably 0.5 to 5% by weight.

Surprisingly and beneficially, dilution of the compound of formula I in the binder has been found to have little or no effect on the charge mobility, in contrast to what would have been expected from the prior art.

The compounds according to the present invention can also be used in mixtures or blends, for example together with other compounds having charge-transport, semiconducting, electrically conducting, photoconducting and/or light emitting semiconducting properties. Thus, another aspect of the invention relates to a mixture or blend comprising one or more compounds of formula I and one or more further compounds having one or more of the above-mentioned properties. These mixtures can be prepared by conventional methods that are described in prior art and known to the skilled person. Typically the compounds are mixed with each other or dissolved in suitable solvents and the solutions combined.

The formulations according to the present invention can additionally comprise one or more further components like for example surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

It is desirable to generate small structures in modern microelectronics to reduce cost (more devices/unit area), and power consumption. Patterning of the layer of the invention may be carried out by photolithography or electron beam lithography.

Liquid coating of organic electronic devices such as field effect transistors is more desirable than vacuum deposition techniques. The formulations of the present invention enable the use of a number of liquid coating techniques.

The organic semiconductor layer may be incorporated into the final device structure by, for example and without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing. The formulations of the present invention are particularly suitable for use in spin coating the organic semiconductor layer into the final device structure.

Ink jet printing is particularly preferred when high resolution layers and devices need to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the mixture of the compound of formula I and the binder should be first dissolved in a suitable solvent. Solvents must fulfil the requirements stated above and must not have any detrimental effect on the chosen print head.

Additionally, solvents should have boiling points >100° C., preferably >140° C. and more preferably >150° C. in order to prevent operability problems caused by the solution drying out inside the print head. Suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a formulation according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the binder and the compound of formula I which reduces or prevents clogging of the jets and separation of the components during spraying. The solvent(s) may include those selected from the following list of examples: dodecylbenzene, 1-methyl-4-tert-butylbenzene, terpineol limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1 to 100 mPa·s, more preferably 1 to 50 mPa·s and most preferably 1 to 30 mPa·s.

The use of the binder in the present invention allows tuning the viscosity of the coating solution, to meet the requirements of particular print heads.

The semiconducting layer of the present invention is typically at most 1 micron (=1 μm) thick, although it may be thicker if required. The exact thickness of the layer will depend, for example, upon the requirements of the electronic device in which the layer is used. For use in an OFET or OLED, the layer thickness may typically be 500 nm or less.

In the semiconducting layer of the present invention there may be used two or more different compounds of formula I. Additionally or alternatively, in the semiconducting layer there may be used two or more organic binders of the present invention.

As mentioned above, the invention further provides a process for preparing the organic semiconducting layer which comprises (i) depositing on a substrate a liquid layer of a formulation which comprises one or more compounds of formula I, one or more organic binders or precursors thereof and optionally one or more solvents, and (ii) forming from the liquid layer a solid layer which is the organic semiconducting layer.

In the process, the solid layer may be formed by evaporation of the solvent and/or by reacting the binder resin precursor (if present) to form the binder resin in situ. The substrate may include any underlying device layer, electrode or separate substrate such as silicon wafer or polymer substrate for example.

In a particular embodiment of the present invention, the binder may be alignable, for example capable of forming a liquid crystalline phase. In that case the binder may assist alignment of the compound of formula I, for example such that their aromatic core is preferentially aligned along the direction of charge transport. Suitable processes for aligning the binder include those processes used to align polymeric organic semiconductors and are described in prior art, for example in US 2004/0248338 A1.

The formulation according to the present invention can additionally comprise one or more further components like for example surface-active compounds, lubricating agents, wetting agents, dispersing agents, hydrophobing agents, adhesive agents, flow improvers, defoaming agents, deaerators, diluents, reactive or non-reactive diluents, auxiliaries, nanoparticles, colourants, dyes or pigments, furthermore, especially in case crosslinkable binders are used, catalysts, sensitizers, stabilizers, inhibitors, chain-transfer agents or co-reacting monomers.

The present invention also provides the use of the semiconducting compound, formulation or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising the formulation according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The compounds and formulations according to the present invention are useful as charge transport, semiconducting, electrically conducting, photoconducting or light mitting materials in optical, electrooptical, electronic, electroluminescent or photoluminescent components or devices. Especially preferred devices are OFETs, TFTs, ICs, logic circuits, capacitors, RFID tags, OLEDs, OLETs, OPEDs, OPVs, OPDs, solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns. In these devices, the compounds of the present invention are typically applied as thin layers or films.

For example, the compound or formulation may be used as a layer or film, in a field effect transistor (FET) for example as the semiconducting channel, organic light emitting diode (OLED) for example as a hole or electron injection or transport layer or electroluminescent layer, photodetector, chemical detector, photovoltaic cell (PVs), capacitor sensor, logic circuit, display, memory device and the like. The compound or formulation may also be used in electrophotographic (EP) apparatus.

The compound or formulation is preferably solution coated to form a layer or film in the aforementioned devices or apparatus to provide advantages in cost and versatility of manufacture. The improved charge carrier mobility of the compound or formulation of the present invention enables such devices or apparatus to operate faster and/or more efficiently.

Especially preferred electronic device are OFETs, OLEDs, OPV devices and OPDs, in particular bulk heterojunction (BHJ) OPV and OPD devices. In an OFET, for example, the active semiconductor channel between the drain and source may comprise the layer of the invention. As another example, in an OLED device, the charge (hole or electron) injection or transport layer may comprise the layer of the invention.

For use in OPV or OPD devices the compounds of formula I according to the present invention are preferably used in a formulation that comprises or contains, more preferably consists essentially of, very preferably exclusively of, a p-type (electron donor) semiconductor and an n-type (electron acceptor) semiconductor. The p-type semiconductor is constituted by one or more compounds of formula I. The n-type semiconductor can be an inorganic material such as zinc oxide (ZnOₓ), zinc tin oxide (ZTO), titan oxide (TiOₓ), molybdenum oxide (MoOₓ), nickel oxide (NiOₓ), or cadmium selenide (CdSe), or an organic material such as graphene or a fullerene or substituted fullerene, for example an indene-C₆₀-fullerene bisaduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J. C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

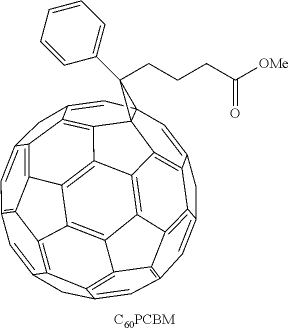 [SMILES: COC(=O)CCCC1(c2ccccc2)C23C=C4Cc5cc6cc7c8c9c(cc%10cc%11c%12c(c%13c%14c%15c(c4c5c4c6c8c(c%154)c4c9c%10c%12c%144)C%1321)C(=C3)C%11)C7]

Preferably the compounds of formula I are blended with an n-type semiconductor such as a fullerene or substituted fullerene, like for example PCBM-C₆₀, PCBM-C₇₀, PCBM-C₆₁, PCBM-C₇₁, bis-PCBM-C₆₁, bis-PCBM-C₇₁, ICBA (1′,1″,4′,4″-tetrahydro-di[1,4]methanonaphthaleno[1,2:2′,3′;56,60:2″,3″][5,6]fullerene-C60-lh), graphene, or a metal oxide, like for example, ZnOₓ, TiOₓ, ZTO, MoOₓ, NiOₓ, to form the active layer in an OPV or OPD device. The device preferably further comprises a first transparent or semi-transparent electrode on a transparent or semi-transparent substrate on one side of the active layer, and a second metallic or semi-transparent electrode on the other side of the active layer.

Further preferably the OPV or OPD device comprises, between the active layer and the first or second electrode, one or more additional buffer layers acting as hole transporting layer and/or electron blocking layer, which comprise a material such as metal oxide, like for example, ZTO, MoOₓ, NiOₓ, a conjugated polymer electrolyte, like for example PEDOT:PSS, a conjugated polymer, like for example polytriarylamine (PTAA), an organic compound, like for example N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), or alternatively as hole blocking layer and/or electron transporting layer, which comprise a material such as metal oxide, like for example, ZnOₓ, TiOₓ, a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly[(9,9-bis(3′-(N,N-dimethylamino)propyl)-2,7-fluorene)-alt-2,7-(9,9-dioctylfluorene)] or an organic compound, like for example tris(8-quinolinolato)-aluminium(III) (Alq₃), 4,7-diphenyl-1,10-phenanthroline.

In a blend or mixture of a compound of formula I with a fullerene or modified fullerene, the ratio compound of formula I versus fullerene is preferably from 5:1 to 1:5 by weight, more preferably from 1:1 to 1:3 by weight, most preferably 1:1 to 1:2 by weight. A polymeric binder may also be included, from 5 to 95% by weight. Examples of binder include polystyrene (PS), polypropylene (PP) and polymethylmethacrylate (PMMA).

To produce thin layers in BHJ OPV devices the compounds or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, dip coating, curtain coating, brush coating, slot dye coating or pad printing. For the fabrication of OPV devices and modules area printing method compatible with flexible substrates are preferred, for example slot dye coating, spray coating and the like.

Suitable solutions or formulations containing the blend or mixture of a compound of formula I with a C₆₀ or C₇₀ fullerene or modified fullerene like PCBM must be prepared. In the preparation of formulations, suitable solvent must be selected to ensure full dissolution of both component, p-type and n-type and take into account the boundary conditions (for example rheological properties) introduced by the chosen printing method.

Organic solvent are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, morpholine, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetraline, decaline, indane, methyl benzoate, ethyl benzoate, mesitylene and combinations thereof.

The OPV device can for example be of any type known from the literature (see e.g. Waldauf et al., Appl. Phys. Lett., 2006, 89, 233517).

A first preferred OPV device according to the invention comprises the following layers (in the sequence from bottom to top):
optionally a substrate,
a high work function electrode, preferably comprising a metal oxide, like for example ITO, serving as anode,
an optional conducting polymer layer or hole transport layer, preferably comprising an organic poymer or polymer blend, for example of PEDOT:PSS (poly(3,4-ethylenedioxythiophene): poly(styrene-sulfonate),
a layer, also referred to as “active layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF or NaF,
a low work function electrode, preferably comprising a metal like for example aluminum, serving as cathode,
wherein at least one of the electrodes, preferably the anode, is transparent to visible light, and
wherein the p-type semiconductor is a compound of formula I.

A second preferred OPV device according to the invention is an inverted OPV device and comprises the following layers (in the sequence from bottom to top):
optionally a substrate,
a high work function metal or metal oxide electrode, comprising for example ITO, serving as cathode,
a layer having hole blocking properties, preferably comprising a metal oxide like ZnOₓ or TiOₓ,
an active layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic poymer or polymer blend, for example of PEDOT:PSS,
an electrode comprising a high work function metal like for example silver, serving as anode,
wherein at least one of the electrodes, preferably the cathode, is transparent to visible light, and
wherein the p-type semiconductor is a compound of formula I.

In the OPV devices of the present invention the p-type and n-type semiconductor materials are preferably selected from the materials, like the OSC/fullerene systems, as described above

When the active layer is deposited on the substrate, it forms a BHJ that phase separate at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morpohology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, chloronaphthalene, and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

The compounds, formulations and layers of the present invention are also suitable for use in an OFET as the semiconducting channel. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound, formulation or organic semiconducting layer according to the present invention. Other features of the OFET are well known to those skilled in the art.

OFETs where an OSC material is arranged as a thin film between a gate dielectric and a drain and a source electrode, are generally known, and are described for example in U.S. Pat. No. 5,892,244, U.S. Pat. No. 5,998,804, U.S. Pat. No. 6,723,394 and in the references cited in the background section. Due to the advantages, like low cost production using the solubility properties of the compounds according to the invention and thus the processibility of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.
   wherein the semiconductor layer preferably comprises a compound of formula I or a formulation as described above and below.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric contant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetry value, like stamps, tickets, shares, cheques etc.

Alternatively, the compounds of formula I can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The compounds of formula I may be employed in one or more of the charge transport layers and/or in the emission layer, corresponding to their electrical and/or optical properties. Furthermore their use within the emission layer is especially advantageous, if the compounds of formula I show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the compounds of formula I, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of the compounds of formula I. Either loss or gain of electrons results in formation of a highly delocalised ionic form, which is of high conductivity.

This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantation of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁻), (NO₂⁺) (BF₄⁻), AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S+(R is an alkyl group).

The conducting form of the compounds of formula I can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and formulations according to the present invention may also be suitable for use in organic plasmon-emitting diodes (OPEDs), as described for example in Koller et al., Nat. Photonics, 2008, 2, 684.

According to another use, the compounds and formulations according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds or materials according to the present invention having mesogenic or liquid crystalline properties can form oriented anisotropic films as described above, which are especially useful as alignment layers to induce or enhance alignment in a liquid crystal medium provided onto said anisotropic film. The materials according to the present invention may also be combined with photoisomerisable compounds and/or chromophores for use in or as photoalignment layers, as described in US 2003/0021913 A1.

According to another use the compounds and formulations according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

The invention will now be described in more detail by reference to the following examples, which are illustrative only and do not limit the scope of the invention.

Above and below, unless stated otherwise percentages are percent by weight and temperatures are given in degrees Celsius. The values of the dielectric constant ∈ (“permittivity”) refer to values taken at 20° C. and 1,000 Hz.

### Example 1

### Example 1.1

### 5-Octyl-[2,2′]bithiophenyl

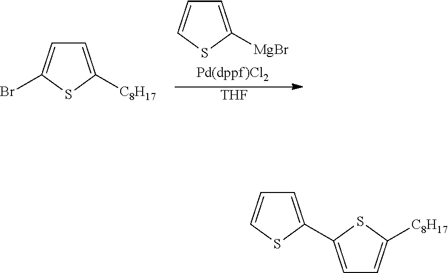

A solution of 2-thienylmagnesium bromide in tetrahydrofuran (1.0 M, 42.5 cm³; 42.5 mmol) is added dropwise, over 30 minutes, to an ice-cooled suspension of 2-bromo-5-octylthiophene (7.8 g; 28 mmol) and [1,1′-bis(diphenylphosphino)ferrocene]dichloropalladium(II) (2.3 g; 2.8 mmol) in anhydrous tetrahydrofuran (125 cm³). The reaction is allowed to warm to 23° C. and stirred for 2 hours. The crude mixture is poured in saturated aqueous solution of ammonium chloride (400 cm³), extracted with ethyl acetate (3×100 cm³), dried over sodium sulfate, filtered and concentrated in vacuo. The crude material is dissolved in dichloromethane (400 cm³), preabsorded onto silica gel (20 g) and purified by column chromatography (silica gel) using petroleum ether 40-60° C. as eluent and recrystallised from petroleum ether 40-60° C. to afford the desired product as off-white crystals (6.0 g, 76%). ¹H NMR (400 MHz, CDCl₃): δ 7.18 (d, J=5.6 Hz, 1H); 7.11 (d, J=3.0 Hz, 1H); 7.02-6.98 (m, 2H); 6.69 (d, J=3.5 Hz, 1H); 2.80 (t, J=7.6 Hz, 2H); 1.74-1.65 (m, 2H); 1.49-1.20 (m, 10H); 0.90 (t, J=7.0 Hz, 3H).

### Example 1.2

### 4-Bromo-5,6-bis-octyloxy-7-thiophen-2-yl-benzo[1,2,5]thiadiazole

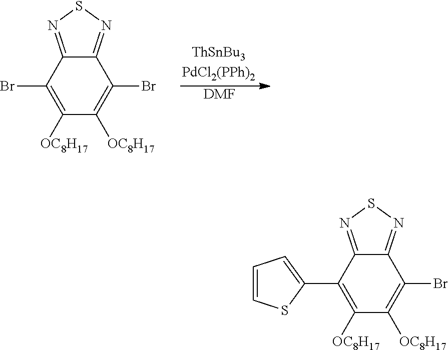

4,7-Dibromo-5,6-bis-octyloxy-benzo[1,2,5]thiadiazole (10 g; 18 mmol), bis(triphenylphosphine)palladium(II) chloride (0.25 g; 0.36 mmol) and tributyl-thiophen-2-yl-stannane (5.7 ml; 18 mmol) are dissolved into degassed dry N,N-dimethylformamide (180 cm³). The reaction mixture is heated to 90° C. for 24 hours under nitrogen. The N,N-dimethylformamide is evaporated in vacuo and the resulting oil dissolved into petroleum ether (50 cm³) and purified by column chromatography (silica gel) twice using a petroleum ether 40-60° C. and dichloromethane mixture (70:30) as eluent to afford the desired product as yellow oil (4.4 g, 44%). ¹H NMR (300 MHz, CDCl₃): δ 8.44 (dd, J=3.8 and 1.1 Hz, 1H); 7.53 (dd, J₁=5.2 Hz, J₂=1.1 Hz, 1H); 7.23 (dd, J₁=5.1 Hz, J₂=3.8 Hz, 2H); 4.20 (t, J=6.7 Hz, 2H); 4.08 (t, J=7.0 Hz, 2H); 1.83-1.98 (m, 4H); 1.20-1.49 (m, 20H); 0.83-0.98 (m, 6H).

### Example 1.3

### 4-(5′-Octyl-[2,2′]bithiophenyl-5-yl)-5,6-bis-octyloxy-7-thiophen-2-yl-benzo[1,2,5]thiadiazole

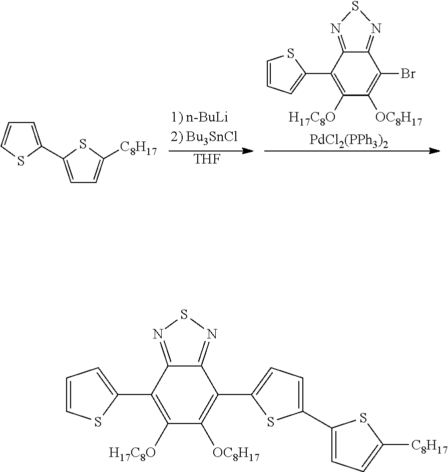

A 2.5 M solution of n-Butyllithium in hexanes (3.5 cm³, 8.7 mmol) is added dropwise over 5 minutes to a suspension of 5-octyl-[2,2′]bithiophenyl (1.1) (2.2 g, 8.0 mmol) in anhydrous tetrahydrofuran (40 cm³) at −70° C. The mixture is stirred for 4 hours at −70° C., then tributyltin chloride (2.4 mL, 8.7 mmol) is added dropwise. The reaction mixture is allowed to warm to 23° C. slowly whilst stirring for 18 hours. 4-Bromo-5,6-bis-octyloxy-7-thiophen-2-yl-benzo[1,2,5]thiadiazole (1.2) (4.0 g, 7.2 mmol) is added and the resulting mixture degassed by ultrasonication for 30 minutes then bis(triphenylphosphine)palladium(II) chloride (0.51 g, 0.72 mmol) added. The reaction is heated to 80° C. for 6 hours, then cooled to 23° C. and the solvent is removed in vacuo. The residue is dissolved in dichloromethane (200 cm³), preabsorded onto silica gel (20 g) and purified by column chromatography (silica gel) using a solvent gradient from 90:10 to 80:20 of petroleum ether 40-60° C. and dichloromethane as eluent to afford the desired product a bright red oil, which solidified on standing (4.5 g, 75%). ¹H NMR (300 MHz, CDCl₃): δ 8.51-8.47 (m, 2H); 7.51 (dd, J₁=5.1 Hz, J₂=1.1 Hz, 1H); 7.26-7.21 (m, 2H); 7.11 (d, J=3.5 Hz, 1H); 6.73 (d, J=3.5 Hz, 1H), 4.17 (t, J=7.0 Hz, 2H), 4.12 (t, J=7.0 Hz, 2H), 2.83 (t, J=7.7 Hz, 2H), 2.04-1.88 (m, 4H), 1.78-1.61 (m, 2H), 1.59-1.20 (m, 30H), 0.99-0.84 (m, 9H).

### Example 1.4

### 4-(5-Bromo-thiophen-2-yl)-7-(5′-octyl-[2,2′]bithiophenyl-5-yl)-5,6-bis-octyloxy-benzo[1,2,5]thiadiazole

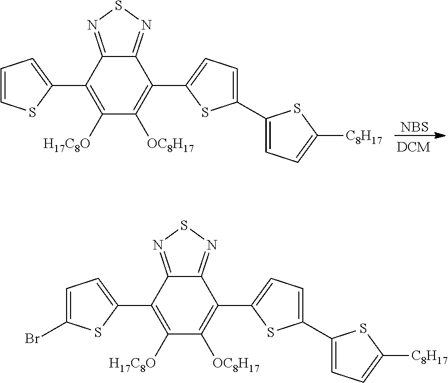

N-Bromosuccinimide (0.28 g, 1.6 mmol) is added to a stirred solution of 4-(5′-Octyl-[2,2′]bithiophenyl-5-yl)-5,6-bis-octyloxy-7-thiophen-2-yl-benzo[1,2,5]thiadiazole (1.3) (1.2 g, 1.6 mmol) in dichloromethane (18 cm³). The reaction mixture is stirred for 18 hours in the dark at 23° C. The crude mixture is diluted with dichloromethane (100 cm³), preabsorbed onto silica gel (3 g) and purified by column chromatography (silica gel) using a 85:15 mixture of petroleum ether 40-60° C. and dichloromethane as eluent to afford the desired product a red oil, which solidified on standing (1.0 g, 75%). ¹H NMR (400 MHz, CDCl₃): δ 8.47 (d, J=4.1 Hz, 1H); 8.37 (d, J=4.1 Hz, 1H); 7.22 (d, J=4.1 Hz, 1H); 7.18 (d, J=4.1 Hz, 1H); 7.11 (d, J=3.5 Hz, 1H); 6.73 (d, J=3.5 Hz, 1H); 4.17 (t, J=7.1 Hz, 2H); 4.13 (t, J=7.1 Hz, 2H); 2.83 (t, J=7.6 Hz, 2H); 2.06-1.90 (m, 4H); 1.79-1.66 (m, 2H); 1.60-1.21 (m, 30H), 1.00-0.86 (m, 9H).

### Example 1.5

### 2,6-Bis-(4,4,5,5-tetramethyl-[1,3,2]dioxaborolan-2-yl)-benzo[1,2-b;4,5-b′]dithiophene-4,8-dicarboxylic acid didodecyl ester

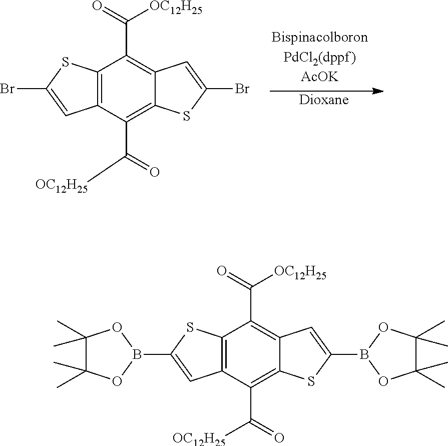

Anhydrous dioxane is degassed for 60 minutes by bubbling nitrogen into the stirred solvent. To a mixture of 2,6-dibromo-benzo[1,2-b;4,5-b′]dithiophene-4,8-dicarboxylic acid didodecyl ester (10 g; 13 mmol), 4,4,5,5,4′,4′,5′,5′-octamethyl-[2,2′]bi([1,3,2]dioxaborolanyl) (7.6 g; 30 mmol), [1,1′-bis(diphenylphosphino)ferrocene]dichloropalladium (11) (1.9 g; 2.3 mmol) and potassium acetate anhydrous (7.6 g; 78 mmol) under nitrogen in a oven dried schlenk tube is added the predegassed anhydrous dioxane (38 cm³). The mixture is then further degassed for 30 minutes and then heated at 80° C. for 17 hours. The mixture is allowed to cool, water (100 cm³) added and the product extracted with dichloromethane (4×150 cm³). The combined organic extracts are dried over anhydrous magnesium sulfate, filtered and the solvent removed in vacuo to give a brown yellow solid. The crude product is purified by multiple hot filtrations in acetonitrile followed by multiple recrystallizations to afford the desired product as yellow needles (4.9 g, 44%). ¹H NMR (300 MHz, CDCl₃): δ 8.79 (s, 2H); 4.58 (t, J=6.7 Hz, 4H); 1.88-1.99 (m, 4H); 1.50-1.61 (m, 4H); 1.34-1.45 (m, 32H); 1.17-1.34 (m, 24H); 0.88 (t, J=6.9 Hz, 6H).

### Example 1.6

### 2,6-Bis-(4-(5-thiophen-2-yl)-7-(5′-octyl-[2,2′]bithiophenyl-5-yl)-5,6-bis-octyloxy-benzo[1,2,5]thiadiazole)-benzo[1,2-b;4,5-b′]dithiophene-4,8-dicarboxylic acid didodecyl ester

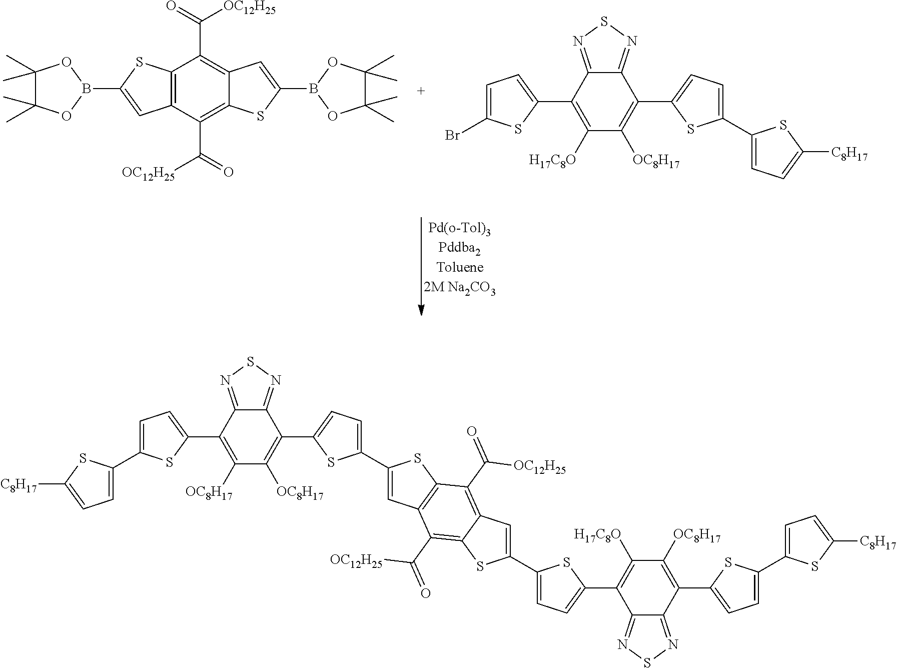

2,6-Bis-(4,4,5,5-tetramethyl-[1,3,2]dioxaborolan-2-yl)-benzo[1,2-b;4,5-b′]dithiophene-4,8-dicarboxylic acid didodecyl ester (1.5) (0.60 g, 0.69 mmol), 4-(5-bromo-thiophen-2-yl)-7-(5′-octyl-[2,2′]bithiophenyl-5-yl)-5,6-bis-octyloxy-benzo[1,2,5]thiadiazole (1.4) (1.2 g, 1.4 mmol), toluene (15 cm³) and a solution of sodium carbonate in water (2.0 cm³, 2.0 M) are degassed by ultrasonication for 30 minutes. Bis(dibenzylideneacetone)palladium (13 mg) and tri(o-tolyl)phosphine (17 mg) are added and the mixture heated to 100° C. (oil bath) for 2 hours. The reaction mixture is poured into water (20 cm³), extracted with dichloromethane (2×50 cm³) and concentrated in vacuo. The crude product is dissolved in dichloromethane (200 cm³), preabsorded onto silica (10 g) and purified several times by column chromatography (silica gel) using a solvent gradient from 70:30 to 50:50 of petroleum ether 40-60° C. and dichloromethane as eluent to afford the desired product as a purple solid (0.48 g, 33%). ¹H NMR (300 MHz, CDCl₃): δ 8.51 (d, J=4.1 Hz, 2H), 8.44 (d, J=4.0 Hz, 2H), 8.38 (s, 2H), 7.45 (d, J=4.1 Hz, 2H), 7.14 (d, J=4.0 Hz, 2H), 7.07 (d, J=3.4 Hz, 2H), 6.70 (d, J=3.6 Hz, 2H), 4.65 (t, J=6.6 Hz, 4H), 4.12-4.26 (m, 8H), 2.81 (t, J=7.6 Hz, 4H), 1.95-2.11 (m, 12H), 1.45-1.74 (m, 20H), 1.14-1.44 (m, 80H), 0.77-0.97 (m, 24H).

### Example 2

### Example 2.1

### 2,6-Bis-(4,4,5,5-tetramethyl-[1,3,2]dioxaborolan-2-yl)-4,8-didodecyl-benzo[1,2-b;4,5-b′]dithiophene

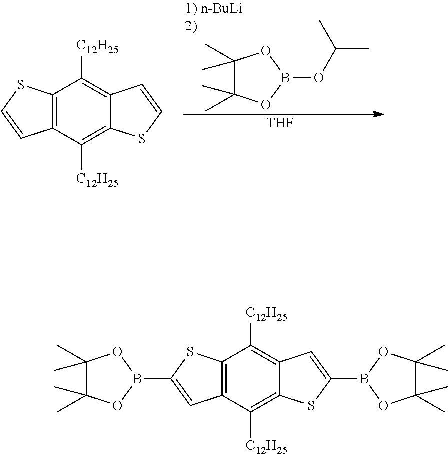

A 2.5 M solution of n-butyllithium (27 cm³, 68 mmol) is added over a period of 5 minutes to a solution of 4,8-didodecyl-benzo[1,2-b;4,5-b′]dithiophene (12 g, 23 mmol) in tetrahydrofuran (500 cm³) at −78° C. The resulting mixture is stirred for 10 minutes at −78° C. and 1 hour at 23° C. The reaction is then cooled to −78° C., 2-isopropoxy-4,4,5,5-tetramethyl-1,3,2-dioxaborolane (16 cm³, 80 mmol) added in one portion and stirred for another 30 minutes at −78° C. and 90 minutes at 23° C. Then, the reaction mixture is poured into water (500 cm³), extracted with diethyl ether (3×200 cm³) and the combined organic extracts further washed with water (200 cm³). The organic phase is removed in vacuo and the residue dissolved in acetone and water is slowly added until a white precipitate is formed. The solid is filtered off and recrystallized twice from acetone to afford the desired product as pale yellow needles (9.9 g, 56%). ¹H NMR (300 MHz, CDCl₃): δ 8.03 (s, 2H) 3.20 (t, J=8.1 Hz, 4H) 1.73-1.86 (m, 4H) 1.38-1.46 (m, 24H) 1.23-1.38 (m, 36H) 0.89 (t, J=6.9 Hz, 6H)

### Example 2.2

### 2,6-Bis-(4-(5-thiophen-2-yl)-7-(5′-octyl-[2,2′]bithiophenyl-5-yl)-5,6-bis-octyloxy-benzo[1,2,5]thiadiazole)-4,8-didodecyl-benzo[1,2-b;4,5-b′]dithiophene

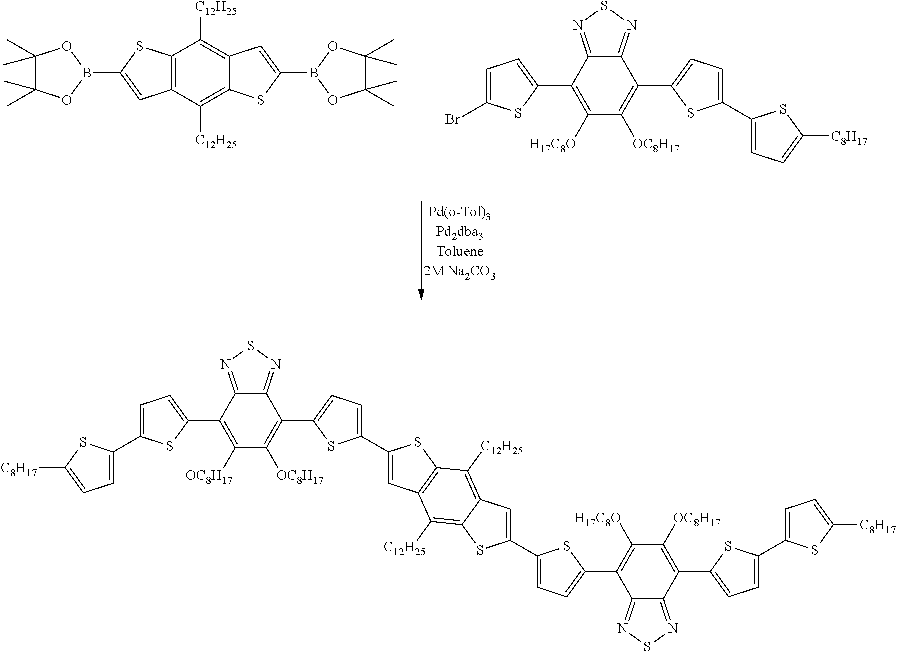

2,6-Bis-(4,4,5,5-tetramethyl-[1,3,2]dioxaborolan-2-yl)-4,8-didodecyl-benzo[1,2-b;4,5-b′]dithiophene (2.1) (0.67 g, 0.86 mmol), 4-(5-bromo-thiophen-2-yl)-7-(5′-octyl-[2,2′]bithiophenyl-5-yl)-5,6-bis-octyloxy-benzo[1,2,5]thiadiazole (1.4) (1.5 g, 1.8 mmol), toluene (20 cm³) and a solution of sodium carbonate in water (2.5 cm³, 2.0 M) are degassed by ultrasonication for 30 minutes. Tris(dibenzylideneacetone)dipalladium (16 mg) and tri(o-tolyl)phosphine (21 mg) are added and the mixture heated to 100° C. (oil bath) for 18 hours. The reaction mixture is poured into water (50 cm³), the organic phase separated and the aqueous further extracted with dichloromethane (3×50 cm³) and the combined organic phases concentrated in vacuo. The crude product is dissolved in dichloromethane (200 cm³), preabsorded onto silica (10 g) and purified several times by column chromatography (silica gel) using a solvent gradient from 90:10 to 75:25 of petroleum ether 40-60° C. and dichloromethane as eluent to afford a purple solid (0.24 g, 14%). ¹H NMR (300 MHz, CDCl₃): δ 8.55 (d, J=4.1 Hz, 2H), 8.50 (d, J=4.1 Hz, 2H), 7.61 (s, 2H), 7.46 (d, J=4.1 Hz, 2H), 7.22 (d, J=4.0 Hz, 2H), 7.11 (d, J=3.5 Hz, 2H), 6.73 (d, J=3.5 Hz, 2H), 4.20 (t, J=6.8 Hz, 4H), 4.18 (t, J=6.8 Hz, 4H), 3.17 (t, J=7.3 Hz, 4H), 2.83 (t, J=7.6 Hz, 4H), 1.92-2.07 (m, 8H), 1.81-1.91 (m, 4H), 1.66-1.76 (m, 4H), 1.55 (s, 16H), 1.17-1.46 (m, 80H), 0.80-0.95 (m, 24H)

### Example 3

Bulk heterojunction organic photovoltaic devices (OPVs) were made using the compounds of Examples 1 and 2.

Organic photovoltaic (OPV) devices are fabricated on pre-patterned ITO-glass substrates (130/sq.) purchased from LUMTEC Corporation.

Substrates are cleaned using common solvents (acetone, iso-propanol, deionized-water) in an ultrasonic bath. A conducting polymer poly(ethylene dioxythiophene) doped with poly(styrene sulfonic acid) [Clevios VPAI 4083 (H. C. Starck)] is mixed in a 1:1 ratio with deionized-water. This solution is filtered using a 0.45 μm filter before spin-coating to achieve a thickness of 20 nm. Substrates are exposed to ozone prior to the spin-coating process to ensure good wetting properties. Films are then annealed at 140° C. for 30 minutes in a nitrogen atmosphere where they are kept for the remainder of the process. Active material solutions (i.e. compound+PCBM-C₆₀) are prepared to fully dissolve the solutes. Thin films are either spin-coated or blade-coated in a nitrogen atmosphere to achieve active layer thicknesses between 50 and 500 nm as measured using a profilometer. A short drying period follows to ensure removal of any residual solvent.

Typically, blade-coated films were dried at 70° C. for 2 minutes on a hotplate. For the last step of the device fabrication, Ca (30 nm)/Al (100 nm) cathodes are thermally evaporated through a shadow mask to define the cells. Current-voltage characteristics are measured using a Keithley 2400 SMU while the solar cells are illuminated by a Newport Solar Simulator at 100 mW·cm⁻² white light. The solar simulator is equipped with AM1.5G filters. The illumination intensity is calibrated using a Si photodiode.

All the device preparation and characterization is done in a dry-nitrogen atmosphere.

Power conversion efficiency (PCE) is calculated using the following expression

$$η = \frac{{V}_{oc} \times {J}_{sc} \times FF}{{P}_{in}}$$

where FF is defined as

$$FF = \frac{{V}_{max} \times {J}_{max}}{{V}_{oc} \times {J}_{sc}}$$

OPV device characteristics for a blend of polymer and fullerene coated from a o-dichlorobenzene solution at a total solid concentration are shown in Table 4.

TABLE 4

| | Unit | Example 1 | Example 2 |
|---|---|---|---|
| Ratio of Compound to | | 1.00:1.50 | 1.00:1.50 |
| PCBM-C₆₀ | | | |
| Concentration | [mg ml⁻¹] | 30 | 30 |
| Voc | [mV] | 150 | 643 |
| Jsc | [mA cm⁻²] | 0.00 | −1.93 |
| FF | [%] | 27.1 | 28.6 |
| PCE | [%] | 0.00 | 0.36 |

1. A compound of formula I
R^{t1}-(Ar¹)ₐ-(Ar²)_{b}-[(Ar³)_{c}-(Ar⁴)_{d}-U-(Ar⁵)ₑ-(Ar⁶)_{f}]ₙ-(Ar⁷)_{g}-(Ar⁸)ₕ-R^{t2} I
wherein
U is a divalent group of the following structure 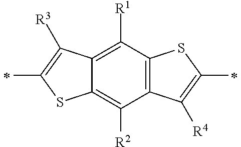
Ar¹⁻⁸ independently of each other denote —CY¹═CY²—, —C≡C—, or aryl or heteroaryl that has 5 to 30 ring atoms and is unsubstituted or substituted by one or more groups R or R¹, and one or more of Ar¹⁻⁸ may also denote U, and wherein those of Ar¹⁻⁸ that are directly adjacent to a group U are different from phenyl and naphthyl,
Y¹, Y² independently of each other denote H, F, Cl or CN,
R¹⁻⁴ independently of each other denote H, F, Cl, —CN, CF₃, R, —CF₂—R, —S—R, —SO₂—R, —C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)—NRR′, —NR′—C(O)—R, —CR′═CR″R′″,
R is alkyl with 1 to 30 C atoms which is straight-chain, branched or cyclic, and is unsubstituted, substituted with one or more F or Cl atoms or CN groups, or perfluorinated, and in which one or more C atoms are optionally replaced by —O—, —S—, —C(O)—, —C(S)—, —SiR⁰R⁰⁰—, —NR⁰R⁰⁰—, —CHR⁰═CR⁰⁰— or —C≡C— such that O- and/or S-atoms are not directly linked to each other,
R⁰, R⁰⁰ independently of each other denote H or C₁₋₁₀ alkyl,
R′, R″, R′″independently of each other have one of the meanings of R or denote H,
R^{t1,t2} independently of each other denote H, F, Cl, Br, —CN, —CF₃, R, —CF₂—R, —O—R, —S—R, —SO₂—R, —SO³—R—C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)NRR′, —NR′—C(O)—R, —NHR, —NRR′, —CR′═CR″R′″, —C≡C—R′, —C≡C—SiR′R″R′″, —SiR′R″R′″, —CH═C(CN)—C(O)—OR, —CH═C(COOR)₂, CH═C(CONRR′)₂, CH═C(CN)(Ar⁹), 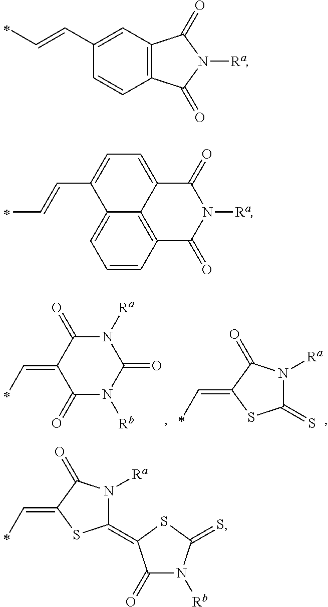
R^{a}, R^{b} are independently of each other aryl or heteroaryl, each having from 4 to 30 ring atoms and being unsubstituted or substituted with one or more groups R or R¹,
Ar⁹ is aryl or heteroaryl, each having from 4 to 30 ring atoms and being unsubstituted or substituted with one or more groups R or R¹,
a-h are independently of each other 0 or 1, with at least one of a-h being 1,
n is 1, 2 or 3.

2. The compound of claim 1, wherein one or more of Ar¹⁻⁸ are selected from aryl or heteroaryl groups having electron acceptor properties.

3. The compound of claim 1, which is selected from the following subformulae 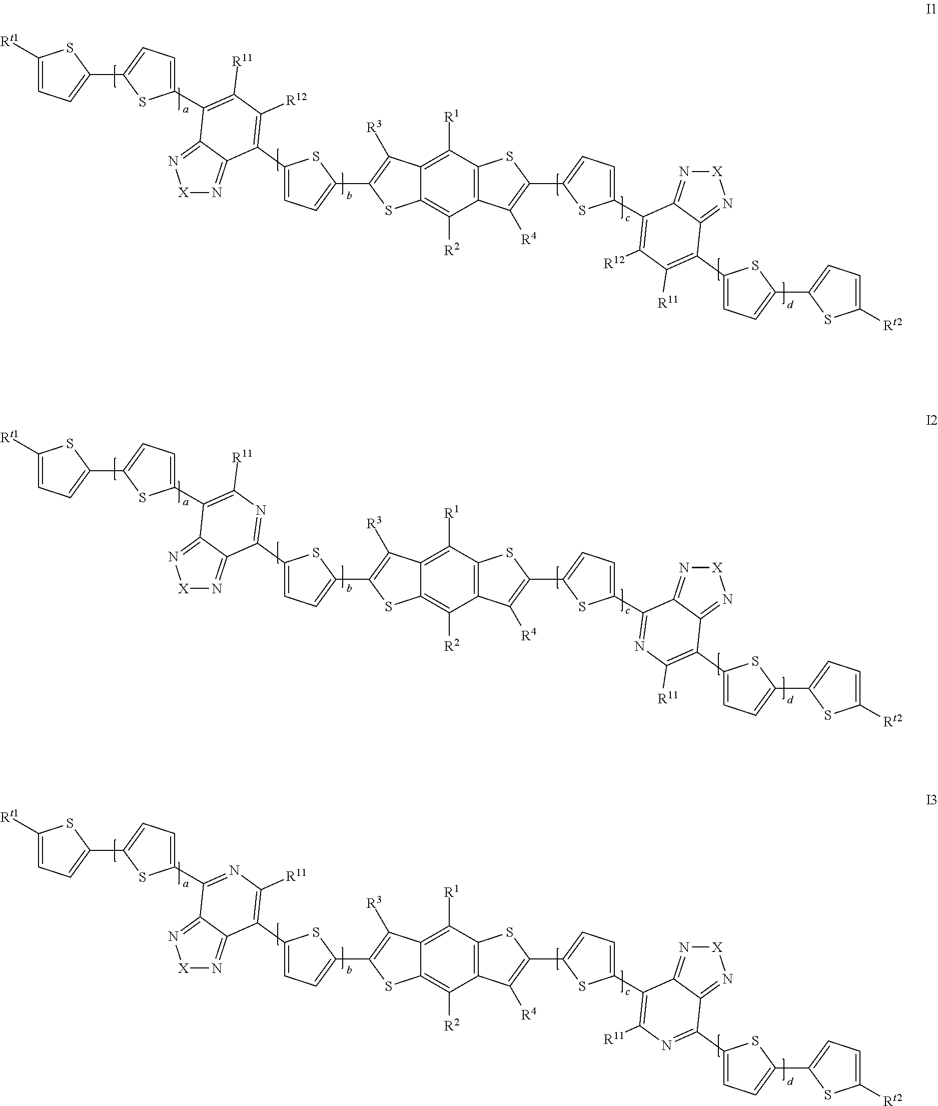 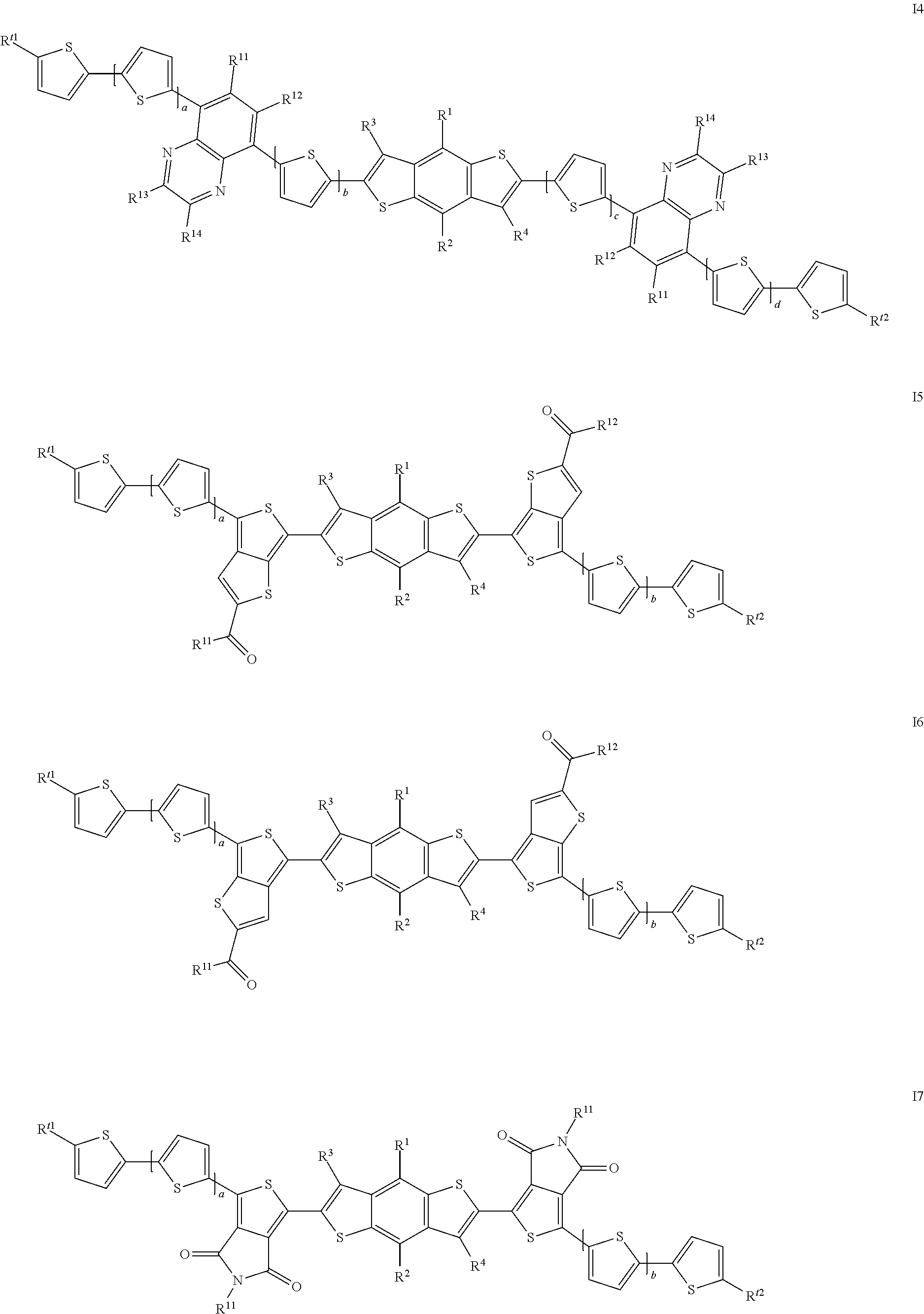 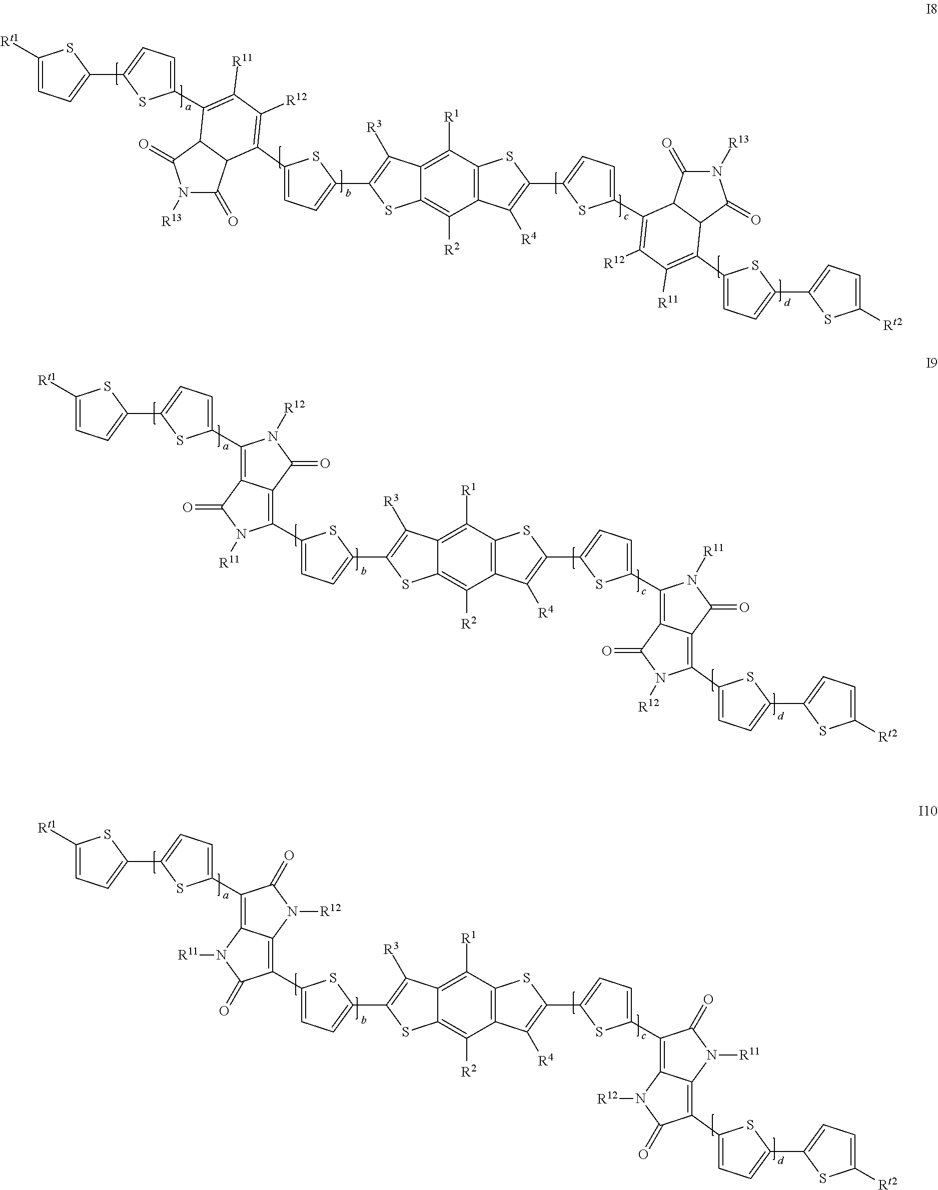 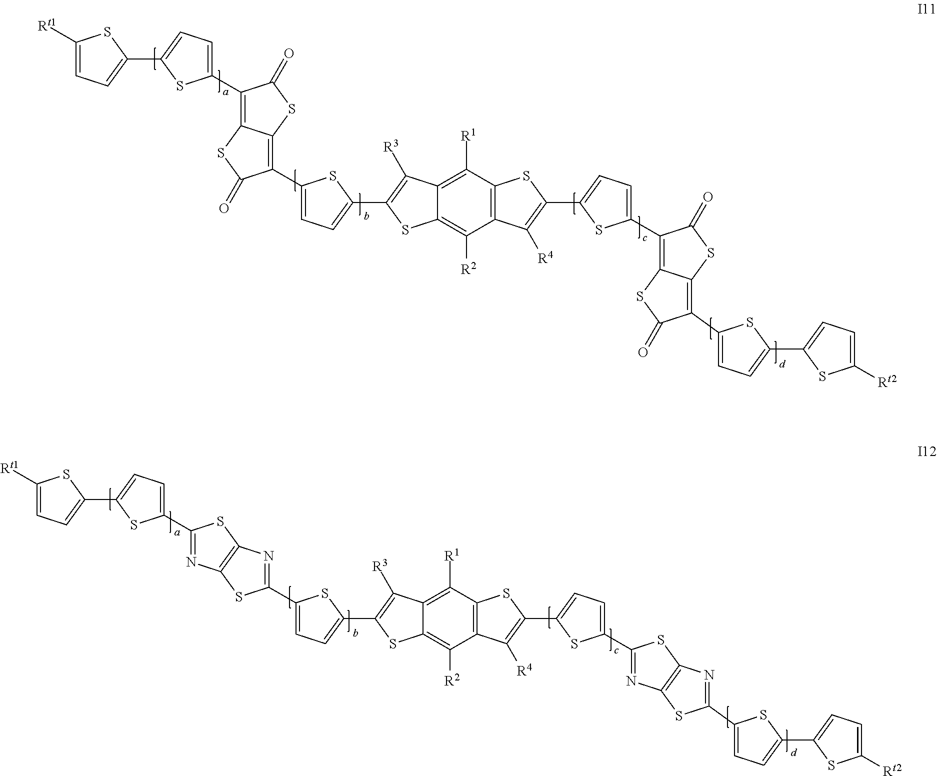
wherein R¹⁻⁴, R^{t1}, R^{t2} have the meanings given in claim 1,
X denotes NR, O, S or Se, with R being as defined in claim 1,
R¹¹⁻¹⁴ have one of the meanings given for R¹, and preferably denote H or alkoxy with 1 to 20 C atoms,
a, b, c and d are 0 or 1, with a+b+c+d≧0, and preferably a=b=c=d=1.

4. The compound according to claim 1, wherein R¹ and R² are independently of each other selected from
the group consisting of primary alkyl with 1 to 30 C atoms, secondary alkyl with 3 to 30 C atoms, and tertiary alkyl with 4 to 30 C atoms, wherein in all these groups one or more H atoms are optionally replaced by F,
the group consisting of primary alkoxy or sulfanylalkyl with 1 to 30 C atoms, secondary alkoxy or sulfanylalkyl with 3 to 30 C atoms, and tertiary alkoxy or sulfanylalkyl with 4 to 30 C atoms, wherein in all these groups one or more H atoms are optionally replaced by F, and
the group consisting of F, Cl, Br, I, CN, —CF₃, —CF₂—R⁹, —C(O)—R⁹, —C(O)—O—R⁹, —O—C(O)—R⁹, —SO₂—R⁹, wherein R⁹ is straight-chain, branched or cyclic alkyl with 1 to 30 C atoms, in which one or more C atoms are optionally replaced by —O—, —S—, —C(O)—, —C(S)—, —NR⁰R⁰⁰—, —CHR⁰═CR⁰⁰— or —C≡C— such that O- and/or S-atoms are not directly linked to each other, and in which one or more H atoms are optionally replaced by F, Cl or CN.

5. A formulation comprising one or more compounds according to claim 1 and one or more organic solvents.

6. The formulation according to claim 5, which further comprises one or more organic binders or precursors thereof, preferably having a permittivity ∈ at 1,000 Hz and 20° C. of 3.3 or less.

7. Use of a compound or formulation according to claim 1 as charge transport, semiconducting, electrically conducting, photoactive, photoconducting or light emitting material in an optical, electrooptical, electronic, electroluminescent or photoluminescent device, or in a component of such a device, or in an assembly comprising such a device or component.

8. A charge transport, semiconducting, photoactive, electrically conducting, photoactive, photoconducting or light emitting material comprising a compound or formulation according to claim 1.

9. An optical, electrooptical, electronic, electroluminescent or photoluminescent device, or a component thereof, or an assembly comprising it, which comprises a charge transport, semiconducting, electrically conducting, photoactive, photoconducting or light emitting material, or comprises a compound or formulation according to claim 1.

10. The optical, electrooptical, electronic, electroluminescent or photoluminescent device according to claim 9, which is selected from organic field effect transistors (OFET), organic thin film transistors (OTFT), organic light emitting diodes (OLED), organic light emitting transistors (OLET), organic photovoltaic devices (OPV), organic photodetectors (OPD), organic solar cells, laser diodes, organic plasmon-emitting diodes (OPEDs), Schottky diodes, organic photoconductors (OPCs) and organic photodetectors (OPDs).

11. The device according to claim 9, which is an OFET, bulk heterojunction (BHJ) OPV device or inverted BHJ OPV device.

12. The component according to claim 9, which is selected from charge injection layers, charge transport layers, interlayers, planarising layers, antistatic films, polymer electrolyte membranes (PEM), conducting substrates and conducting patterns.

13. The assembly according to claim 9, which is selected from integrated circuits (IC), radio frequency identification (RFID) tags or security markings or security devices containg them, flat panel displays or backlights thereof, electrophotographic devices, electrophotographic recording devices, organic memory devices, sensor devices, biosensors and biochips.

14. A compound of formula II
R⁵-(Ar¹⁰)ᵢ-U-(Ar¹¹)ₖ-R⁶ II
wherein U is as defined in claim 1,
Ar¹⁰, Ar¹¹ independently of each other, and on each occurrence identically or differently, have one of the meanings of Ar as given in claim 1,
i, k are independently of each other 0, 1, 2 or 3, with i+k>0, and
R⁵, R⁶ are independently of each other a leaving group, preferably selected from the group consisting of H, F, Br, Cl, I, —CH₂Cl, —CHO, —CR^{a}═CR^{b}2, —SiR^{a}R^{b}R^{c}, —SiR^{a}X′X″, —SiR^{a}R^{b}X′, —SnR^{a}R^{b}R^{c}, —BR^{a}R^{b}, —B(OH)₂, —B(OZ²)₂, —O—SO₂Z, O-tosylate, O-triflate, O-mesylate, O-nonaflate, —SiMe₂F, —SiMeF₂, —CZ³═C(Z³)₂, —C≡CH, —C≡CSi(Z¹)₃, —ZnX′ and —Sn(Z⁴)₃, wherein X′ and X″ denote halogen, preferably Cl, Br or I, R^{a}, R^{b} and R^{c} independently of each other denote H or alkyl with 1 to 20 C atoms, two of R^{a}, R^{b} and R^{c} may also form an aliphatic ring together with the hetero atom to which they are attached, Z¹⁻⁴ are selected from the group consisting of alkyl and aryl, each being optionally substituted, and two groups Z² may also together form a cyclic group.
